(19) United States
(12) Patent Application Publication
(10) Pub. No.: US 2020/0289636 A1
(43) Pub. Date: Sep. 17, 2020

(54) FILOVIRUS VACCINES AND METHODS OF USE

(71) Applicant: Hawaii Biotech, Inc., Honolulu, HI (US)

(72) Inventors: David E. CLEMENTS, Honolulu, HI (US); Michael M. LIEBERMAN, Honolulu, HI (US); Axel T. LEHRER, Honolulu, HI (US)

(21) Appl. No.: 16/645,417

(22) PCT Filed: Sep. 6, 2018
(86) PCT No.: PCT/US2018/049769

§ 371 Date: Mar. 6, 2020

Related U.S. Application Data
(60) Provisional application No. 62/555,543, filed on Sep. 7, 2017.

(51) Int. Cl. A61K 39/12; A61P 31/14; A61K 39/295

(52) U.S. Cl. CPC A61K 39/12; A61K 2039/55583; A61K 39/295; A61P 31/14

(57) ABSTRACT

The data reported herein describe the production and evaluation of a recombinant subunit filovirus vaccine using insect cell expressed surface glycoprotein (GP) and a highly effective adjuvant. The vaccine provides protection in humans against filovirus infection, including Ebola virus and Marburg virus.

### CROSS-REFERENCE TO RELATED APPLICATIONS

This application claims benefit of priority under 35 U.S.C. § 119(e) of U.S. Ser. No. 62/555,543, filed Sep. 7, 2017, the entire contents of which is incorporated herein by reference in its entirety.

### GOVERNMENT SUPPORT

This invention was made with government support under AI119185 and AI32323 awarded by the National Institutes of Health. The government has certain rights in the invention.

### BACKGROUND OF THE INVENTION

### Field of the Invention

The invention relates generally to vaccines and more specifically to a recombinant non-replicating vaccine for filoviruses, including Ebola Virus and Marburg Virus.

### Background Information

Although the frequency of human infections is low, the extreme virulence of filoviruses has heightened both public and scientific awareness. The most prominent members of the family are Zaire ebolavirus (EBOV) and Marburg marburgvirus (MARV) which cause fulminant hemorrhagic fevers and death in up to 90% of human infections depending on the infecting strain, route of infection and medical care provided. While state of the art medical treatment may increase the chances of survival after EBOV infection, currently no vaccine or antiviral therapy is available to prevent or cure the disease. As shown during the West African outbreak of EBOV (2013-2016), diagnostic capabilities as well as the required supportive treatment of patients is very resource demanding and therefore the development of safe and effective prophylactic vaccines is very important in preventing and combating future outbreaks. As part of the outbreak response in the affected West African countries, WHO and various industrial and government partners collaborated on expedited clinical paths for EBOV vaccines and therapeutics. The most promising reports on progress towards an efficacious EBOV vaccine have been of human clinical trials of a recombinant replication-competent Vesicular Stomatitis Virus (VSV) vectored Ebola vaccine containing the EBOV GP protein in place of the VSV G protein. The efficacy and effectiveness of this vaccine (rVSV-ZEBOV) was assessed in a phase 3 clinical trial using the approach of ring-vaccinations in Guinea, West Africa. The interim and final reports showed that a single administration of the vaccine was efficacious and effective and deemed safe as well which led to recent (December 2016) public statements by the WHO declaring the vaccine trial to be successful.

Indeed, the results of the ring-vaccination, cluster randomized trial demonstrated that the vaccine efficacy was 100% based on the occurrence of new cases of Ebola Virus Disease (EVD) more than ten days after identification of an index case when comparing results from immediate-versus delayed vaccinated trial subjects (primary and secondary contacts of EVD index cases). The occurrence of EVD cases during the first nine days after identification of the cluster was not different between the two study groups. While these developments are encouraging and seem to provide a viable path to market for the first EBOV vaccine candidate, many hurdles, particularly in regards to safety, stability, and durability of protection remain to be overcome. In contrast to many other viral infections, the pathology of filovirus hemorrhagic fevers in primate hosts is not linked to systemic viremia, but to a dysregulation of the immune system. Thus, disease pathogenesis should also be viewed from an immunological perspective.

An understanding of critical virus-host interactions that lead to development of a protective adaptive immune response instead of lymphocytopenia, thrombocytopenia, hemorrhage and death is essential for developing immune therapeutics or prophylactic vaccines. One possible link to EVD survival may be the kinetics of the host's immune response. For humoral responses, faster immunoglobulin class switching in human convalescents compared to casualties in the Kikwit outbreak (1995) of EBOV has been described as well as the more rapid development of cellular immunity. Whole blood transfer from human convalescents seemed to improve the outcome for treated patients. These observations and the fact that non-human primate (NHP) survivors of EBOV challenge are immune to subsequent EBOV infection, suggest that prophylactic vaccination is possible. In a recent report from a human clinical trial of the “rVSV-ZEBOV” vaccine candidate described by Khurana et al., the investigators demonstrate that the human antibody profile generated by this vaccine consists largely of IgM isotype antibody, with a lack of antibody class switching and affinity maturation. Furthermore, the antibody titers appear to decline rapidly after vaccination with only about 10-20% of peak titers remaining 84 days post vaccination and no apparent booster effect after another dose of vaccine. While the IgM antibodies demonstrated activity in a pseudovirion neutralization assay, their avidity was relatively low. This raises questions about the durability of protection afforded by this vaccine candidate and warrants further research into vaccine immunogenicity and potential prime-boost approaches.

Filoviruses are enveloped, negative strand RNA viruses. The viral RNA is packaged with viral nucleoprotein (NP) and the envelope is formed by the association of the viral matrix proteins VP40 and VP24 with the membrane containing the mature surface glycoprotein (GP). GP has been identified as the viral protein leading to cell surface binding and membrane fusion and has therefore been selected as the major candidate antigen which may also induce virus neutralizing antibodies, even though different mechanisms other than classical virus neutralization such as antibody dependent cytotoxicity or cell-mediated immunity may also be required to clear EBOV infections. Several preclinical challenge studies have demonstrated that immune responses to EBOV GP raised with various experimental approaches using viral vectors (VSV, various adenoviruses, or human parainfluenza virus (HPIV)) may be sufficient to protect NHP against death from EBOV infection. The use of additional viral proteins (e.g., VP24, VP40, or NP) may contribute to vaccine efficacy and possibly also to the cross-protective potential of a candidate vaccine since they are more conserved amongst different filoviruses than the GPs.

The cross-protective potential of additional virus proteins was shown indirectly in a comparative experiment in guinea pigs in which groups of animals were vaccinated with recombinant VSV vectors expressing only the GPs of EBOV, Sudan ebolavirus (SUDV), Tai Forest ebolavirus (TAFV) or Reston ebolavirus (RESTV) or immunized by infection with the four wild-type (non-guinea pig adapted) ebolavirus species which are non-lethal to guinea pigs. Only recipients of the recombinant VSV vaccine expressing EBOV GP were protected against challenge with guinea-pig adapted EBOV while animals immunized with the GPs of SUDV, TAFV, or RESTV succumbed to disease. In contrast, animals “immunized” by infection with each of the four non-adapted ebolaviruses were protected against lethal challenge with guinea pig-adapted EBOV independent of the species used for vaccination. This suggests that the cross-protective potential must be found in adaptive responses raised by viral component(s) other than GP. One of these potential vaccine candidate antigens is NP which has been utilized in DNA vaccinations, adenovirus-vectored approaches and as part of virus-like particle (VLP) vaccine development efforts. NP is abundantly present in mature virions as it forms the nuclear core together with genomic RNA and has been shown to possess T-cell epitopes. Studies have shown that Venezuelan Equine Encephalitis virus replicon particles (VRP) expressing NP can elicit cytotoxic T-cell responses in mice. The matrix protein VP40, a major component of the virus particle, and the minor matrix protein VP24 are possible additional vaccine antigens. Both have shown protective potential in mouse challenge studies when administered in the form of VRPs. Subsequent work showed that VRPs expressing VP24 or VP40 induce cytotoxic T lymphocytes(CTL) that confer protection in mice.

Multiple filovirus vaccine candidates employing recombinant technologies have demonstrated promise in preclinical studies; however, thus far the mechanisms by which the virus components induce protection are unknown. As expected, the GP has proven useful as a vaccine antigen in animals, including NHP, using recombinant VSV, HPIV or adenoviruses as vectors. Recombinant protein antigens in the form of VLP's produced in mammalian or insect cells have also been shown to induce protection in rodents and NHP. In contrast to the recombinant EBOV and MARV VLP's, inactivated MARV and EBOV induced only partial protection in NHPs. These results may be related to the structural damage caused by denaturation during irradiation of the viruses. The lack of efficacy may also be caused by incorrect presentation and/or processing of antigens, incorrect dosing, use of inadequate adjuvants, or due to contaminating proteins.

Achieving proper conformation of complex viral proteins is often problematic and the Drosophila S2 expression system has demonstrated the ability to overcome the challenges and produce conformationally relevant envelope proteins for a number of viral vaccine targets. The native-like structure of dengue envelope proteins produced in this manner has been demonstrated through the determination of X-ray crystal structures. In contrast to virally vectored vaccines, DNA-vaccines or virus-like particles, formulations of recombinant subunits allow for delivery of well-defined antigen combinations that are designed to achieve optimal safety and potency in diverse populations. Therefore, a detailed understanding of the mechanism by which protective responses are achieved with the individual antigens is required.

### SUMMARY OF THE INVENTION

The present invention relates to new vaccines and, in particular, filovirus vaccines. The invention is based on the seminal discovery of a filovirus vaccine that protects humans against pathogenic filoviruses, including Zaire Ebolavirus (EBOV), Sudan Ebolavirus (SUDV) and Marburgvirus (MARV). The inventors have developed vaccines, including multicomponent vaccine formulations composed of highly purified subunit proteins that provide potent efficacy against filovirus infection in a primate model that is widely accepted in the art as predictive of the effect in humans.

In one aspect, one adjuvant was found to be highly effective when formulated with the purified filovirus subunit proteins. This adjuvant includes sucrose fatty acid sulphate esters (SFASE) immobilized on the oil droplets of a submicron emulsion of squalane-in-water (Blom A G, Hilgers L A (2004) Sucrose fatty acid sulphate esters as novel vaccine adjuvants: effect of the chemical composition. Vaccine 23: 743-754). In one illustrative example, the sucrose fatty acid ester adjuvant is CoVaccine HT™.

In one embodiment, the invention provides an immunogenic composition comprising at least one filovirus glycoprotein (GP) formulated with a sucrose fatty acid sulphate ester, wherein the composition elicits an immune response when administered to a subject, which response is protective upon challenge with a filovirus. In another embodiment, the composition further comprises at least one matrix protein. For example, the matrix protein may include VP24 and/or VP40 as disclosed herein in an illustrative example of a vaccine of the invention.

In one aspect, the glycoproteins are from EBOV. In one aspect, the glycoproteins are from MARV.

In one embodiment, the invention provides a method of inducing a protective immune response to infection with a filovirus comprising administering to a subject in need thereof, a protective effective amount of a composition including at least one filovirus glycoprotein (GP) formulated with a sucrose fatty acid sulphate ester, thereby protecting the subject from infection with the filovirus. In one illustrative example, the sucrose fatty acid ester adjuvant is CoVaccine HT™. The vaccine of the invention is particularly suited for use in humans. In one aspect, the glycoproteins are from EBOV. In one aspect, the glycoproteins are from MARV.

### BRIEF DESCRIPTION OF THE DRAWINGS

FIGS. 1A-1B show antibody responses in rhesus macaques following immunization and challenge. Panel A: anti-GP IgG titers. Panel B: anti-VP24 IgG titers. Day 0 is the first vaccination day followed by two boosters on days 21 and 42. Challenges occurred on day 71. Formulation 1: 50 μg each of GP and VP24+alum; Formulation 2: 25 μg each of GP and VP24, 5 μg VP40+CoVaccine HT; Formulation 3: Alum control. Survivors: RHK61 (green), RHG88 (green), RHJRD (black).

FIG. 2A shows IgG titers against EBOV GP raised by three doses of candidate vaccines. CoVaccine HT containing formulation UHM-1 is indicated in blue (animals 18014 and BB206F). 18266, 18272 received UHM-2 and 16984, 26311 received UHM-3.

FIG. 2B (left) and FIG. 2C (right) show IgG titers against EBOV VP40 and VP24, respectively, raised by three doses of candidate vaccines. UHM-1 is indicated in blue (animals 18014 and BB206F). 18266, 18272 received UHM-2 and 16984, 26311 received UHM-3.

FIG. 3 shows the survival of vaccinated and control monkeys after EBOV challenge. Using either the Log-rank (Mantel-Cox) test or the Gehan-Breslow-Wilcoxon test, both of the curves for the vaccinated animals are significantly different from the controls (p=0.0082).

FIG. 4 shows the kinetics of viremia for 14 days after challenge. Viremia was determined by rt-PCR on serum samples from individual animals—Limit of detection: 3 log₁₀. The data demonstrate the inhibition of viremia as a result of vaccination with UHM-4. The animals vaccinated with UHM-1 showed slightly higher virus load than animals vaccinated with GP+CoVaccine HT.

FIG. 5 shows results of a viremia post challenge by plaque assay. Viral plaques were only observed in both control animals, and with a significant delay in one animal immunized with the formulation UHM-1. This demonstrates dramatically how vaccination with the recombinant subunit monovalent Ebola vaccine completely protects all vaccinees from infection with Ebola Virus.

FIG. 6 shows IgG antibody titers to Ebola GP antigen determined by the MIA assay on vaccinated animals. Animals were immunized on days 0, 21, and 42. Antibody levels in vaccinated animals rose rapidly after the first and second immunizations and reached a plateau by 14 days post dose 2 (day 35).

FIG. 7 shows antibody titers in mice vaccinated with liquid and lyophilized antigens after incubation at elevated temperatures.

FIG. 8 shows non-human primate survival after vaccination with EBOV GP and challenge with live EBOV (low passage 7U variant of the Kikwit strain).

FIG. 9 shows non-human primate survival after vaccination with MARV GP or MARV+EBOV GP (BiFiloVax liquid) and challenge with live MARV (low passage Angola strain).

FIG. 10 is a graph showing anti-EBOV GP IgG at various time points post vaccination.

FIG. 11 is a graph showing anti-MARV GP IgG at various time points post vaccination.

FIG. 12 is an SDS-PAGE gel showing expression of recombinant EBOV subunits from Drosophila S2 cells.

FIG. 13 is a gel showing purified recombinant EBOV proteins.

FIG. 14 is an SDS-PAGE gel showing the glycosylation status of recombinant EBOV GP.

FIG. 15 is an SDS-PAGE gel showing the glycosylation status of recombinant EBOV VP40.

FIG. 16 is a MALDI-Tof analysis of recombinant EBOV VP40.

FIG. 17 is a MALDI-Tof analysis of recombinant EBOV VP24.

FIG. 18 is a graph showing humoral responses to recombinant EBOV antigens.

FIGS. 19A-19C show graphs for cell-mediated immune responses raised by recombinant antigens.

FIGS. 20A-20D show graphs depicting humoral responses based on adjuvant selection and antigen dose.

FIGS. 21A-21C show graphs for antibody titration curves for mice immunized with GP, VP24 or VP40 with either GPI-0100 or ISA51 adjuvants.

FIGS. 22A-22B are graphs showing Kaplan-Meier survival plots of actively and passively immunized and challenged mice.

FIG. 23 is a graph showing weight change after challenge in actively or passively immunized mice and control mice.

FIG. 24 shows ELISA IgG antibody titers to irradiated whole virus after 3 immunizations and prior to virus challenge.

FIGS. 25A-25B show grpahs of IgG Elisa antibody titers (EC50) against recombinant EBOV GP and VP40.

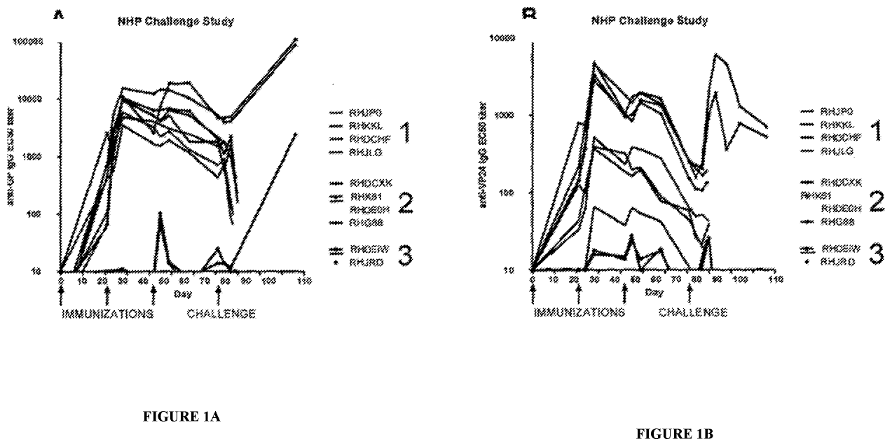

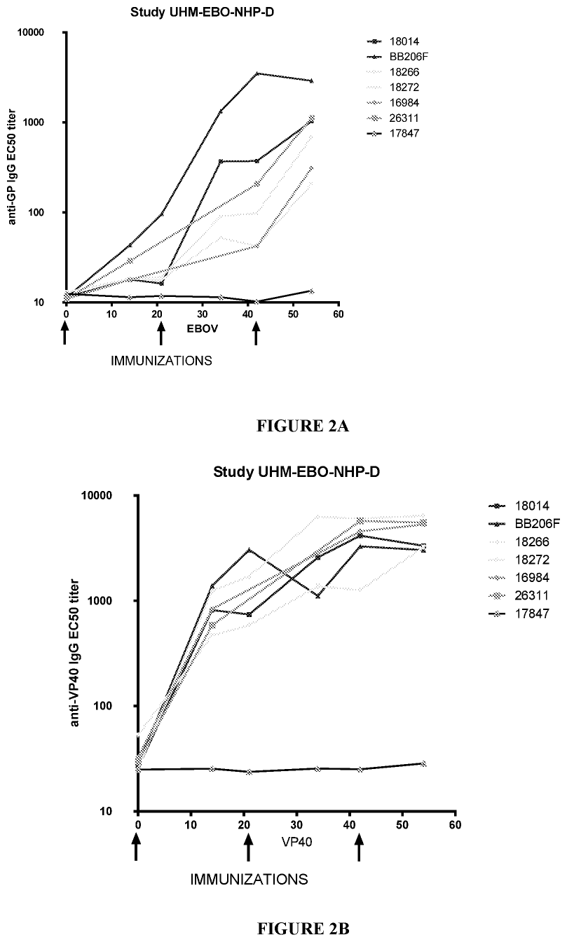

### DETAILED DESCRIPTION OF THE INVENTION

Infections with filoviruses in humans are highly virulent, causing hemorrhagic fevers which result in up to 90% mortality. Currently, there are no licensed vaccines or therapeutics available to combat these infections. The pathogenesis of disease involves the dysregulation of the host's immune system, which results in impairment of the innate and adaptive immune responses, with subsequent development of lymphopenia, thrombocytopenia, hemorrhage, and death.

Questions remain regarding the few survivors of infection, who manage to mount an effective adaptive immune response. These questions concern the humoral and cellular components of this response, and whether such a response can be elicited by an appropriate prophylactic vaccine. The data reported herein describe the production and evaluation of a recombinant subunit Ebola virus vaccine candidate and a Marburg virus vaccine candidate which include insect cell expressed Zaire ebolavirus (EBOV) surface glycoprotein (GP) or Marburg virus surface glycoprotein. In some aspects, the EBOV vaccine may include the matrix proteins VP24 and/or VP40. Thus, the invention provides monovalent, bivalent, trivalent or other vaccine formulations.

The recombinant subunit proteins are shown to be highly immunogenic in mice and non-human primates, yielding both humoral and cellular responses. Furthermore, these vaccine formulations were found to be highly efficacious, providing up to 100% protection against a lethal challenge with live virus in both mice and primates. These results demonstrate proof of concept for a filovirus recombinant non-replicating vaccine candidate for use to protect humans disease caused by filovirus infections such as EBOV and MARV.

In one embodiment, the invention provides a composition comprising at least one filovirus glycoprotein (GP) formulated with an adjuvant, wherein the adjuvant comprises a sucrose fatty acid sulphate ester, wherein the composition elicits an immune response when administered to a subject, which response is protective upon challenge with a filovirus. In some aspects the filovirus is a Zaire Ebolavirus (EBOV), Sudan Ebolavirus (SUDV) or Marburgvirus (MARV).

In one aspect, the adjuvant comprises a physiological salt solution, or an oil-in-water emulsion, or a water immiscible solid phase, and optionally an aqueous phase, and comprising, as an adjuvant, one or more disaccharide derivatives of formula:

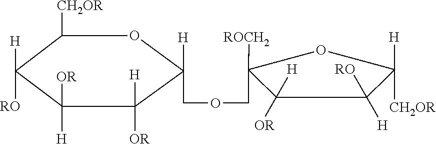

wherein (i) at least 3, but not more than N−1, of the groups R are represented by: —C(═O)—(CH₂)ₓCH₃ groups, wherein x is between 6 and 14, and (ii) at least one, but no more than N−1, of the groups R are anionic —SO₂—OR¹ groups, wherein R¹ is a monovalent cation, wherein N is the number of groups R of the disaccharide derivative and wherein the combined number of —C(═O)—(CH₂)ₓCH₃ and —SO₂—OR¹ groups does not exceed N and the remaining groups R are hydrogen. In one aspect, the disaccharide derivative has no more than N−2, or no more than N−3, anionic —SO₂—OR¹ groups. In one aspect, the disaccharide derivative has at least 4, but no more than N−1, —C(═O)—(CH₂)ₓCH₃ groups and no more than N−3, or no more than N−4, anionic —SO₂—OR¹ groups. In another aspect, the disaccharide derivative has two, three or four anionic —SO₂—OR¹ groups, and at least three —C(═O)—(CH₂)ₓCH₃ groups, wherein the total sum of anionic —SO₂—OR¹ groups and —C(═O)—(CH₂)ₓCH₃ groups is in the range of about 6 or 7.

In one aspect, the monovalent cation is independently selected from the group consisting of H⁺, K⁺, Na⁺, Li⁺ and NH₄⁺. In one aspect, the composition comprises an oil in water emulsion, wherein said oil-in-water emulsion comprises a water-immiscible liquid phase which is squalane, a mineral oil, a plant oil, hexadecane, a fluorocarbon or a silicon oil. In one aspect, the composition further includes an emulsifier or stabilizer. Examples of such emulsifier or stabilizer is a non-ionic detergent with a hydrophilic-lipophilic balance value of more than 10, a sugar fatty acid ester, or an anionic detergent with a hydrophilic-lipophilic balance value of more than 10. Further, the emulsifier or stabilizer may be a disaccharide derivative.

In one aspect, the water immiscible solid phase is an insoluble salt. For example, the insoluble salt is an aluminum or calcium salt, preferably an aluminum hydroxide, aluminum phosphate, calcium phosphate, silica or a mixture thereof. In an illustrative example, the adjuvant is CoVaccineHT™.

The composition of the invention may further include at least one matrix, for example, VP24 and/or VP40.

In one embodiment, the invention provides a method of inducing a protective immune response to infection with a filovirus comprising administering to a subject in need thereof, a protective effective amount of a composition of the invention, thereby protecting the subject from infection with the filovirus. Preferably the subject is a human. Upon administration, the subject develops antibody titers such as IgG or IgM.

In one aspect, administration is in one or more immunizations. In one aspect, the adjuvant is as described above, and comprises a physiological salt solution, or an oil-in-water emulsion, or a water immiscible solid phase, and optionally an aqueous phase, and comprising, as an adjuvant, one or more disaccharide derivatives of formula:

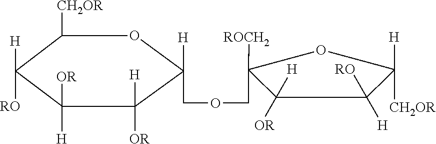

wherein (i) at least 3, but not more than N−1, of the groups R are represented by: —C(═O)—(CH₂)ₓCH₃ groups, wherein x is between 6 and 14, and (ii) at least one, but no more than N−1, of the groups R are anionic —SO₂—OR¹ groups, wherein R¹ is a monovalent cation, wherein N is the number of groups R of the disaccharide derivative and wherein the combined number of —C(═O)—(CH₂)ₓCH₃ and —SO₂—OR¹ groups does not exceed N and the remaining groups R are hydrogen. In particular, the adjuvant may be CoVaccineHT™.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

As used in this specification and the appended claims, the singular forms “a”, “an”, and “the” include plural references unless the context clearly dictates otherwise. Thus, for example, references to “the method” includes one or more methods or steps of the type described herein, which will become apparent to persons skilled in the art upon reading this disclosure.

The term “about” or “approximately” are defined as being close to as understood by one of ordinary skill in the art, and in one non-limiting embodiment the terms are defined to be within 10%, preferably within 5%, more preferably within 1%, and most preferably within 0.5% of the qualified value.

The term “substantially” and its variations are defined as being largely but not necessarily wholly what is specified as understood by one of ordinary skill in the art, and in one non-limiting embodiment substantially refers to ranges within 10%, within 5%, within 1%, or within 0.5% of the qualified value.

The term “effective” as that term is used in the specification and/or claims, means adequate to accomplish a desired, expected, or intended result.

By “pharmaceutically acceptable” it is meant that the carrier, diluent or excipient must be compatible with the other ingredients of the formulation and not deleterious to the recipient thereof. Pharmaceutically acceptable carriers, excipients or stabilizers are well known in the art, for example from Remington's Pharmaceutical Sciences, 16th edition, Osol, A. Ed. (1980). Pharmaceutically acceptable carriers, excipients, or stabilizers are nontoxic to recipients at the dosages and concentrations employed, and may include buffers such as phosphate, citrate, and other organic acids; antioxidants including ascorbic acid and methionine; preservatives such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol; low molecular weight (less than about 10 residues) polypeptides; proteins such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes such as Zn-protein complexes; non-ionic surfactants such as TWEEN™, PLURONICS™, or polyethylene glycol (PEG); or combinations thereof.

The compounds of the present invention can exist as therapeutically acceptable salts. The present invention includes compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002), the entire contents of which are herein incorporated by reference.

The terms “administration of” and “administering a” compound should be understood to mean providing a compound of the disclosure or pharmaceutical composition to a subject. An exemplary administration route is intravenous administration. In general, administration routes include but are not limited to intracutaneous, subcutaneous, intravenous, intraperitoneal, intraarterial, intrathecal, intracapsular, intraorbital, intracardiac, intradermal, transdermal, transtracheal, subcuticular, intraarticulare, subcapsular, subarachnoid, intraspinal and intrasternal, oral, sublingual buccal, rectal, vaginal, nasal ocular administrations, as well infusion, inhalation, and nebulization. The phrases “parenteral administration” and “administered parenterally” as used herein means modes of administration other than enteral and topical administration. The compositions of the present invention may be processed in a number of ways depending on the anticipated application and appropriate delivery or administration of the pharmaceutical composition. For example, the compositions may be formulated for injection.

The compounds can be administered in various modes, e.g. orally, topically, or by injection. In some embodiments, the compounds are administrated by injection. The precise amount of compound administered to a patient can be determined by a person of skill in the art. The specific dose level for any particular patient will depend upon a variety of factors including the activity of the specific compound employed, the age, body weight, general health, sex, diets, time of administration, and route of administration.

The term “subject” as used herein refers to any individual or patient to which the subject methods are performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

The antigens can be used before an infection, for example to protect against future infection. This is similar to a conventional vaccination strategy. Initially stimulated innate immune response provides quick protection, while a subsequent adaptive immune response further protects against the ongoing or subsequent infections. The antigens/compositions can also be used post-infection, to provide additional immunity against an infection. Furthermore, the compositions can also be used to protect against non-infectious conditions, such as cancer. Because the compositions boost an innate immune response (and not only an adaptive one), they are beneficial against non-infectious conditions as well. This makes their use broader than what the source of the antigen(s) may indicate. As such, their use is not limited to filovirus infections.

The following examples are provided to further illustrate the embodiments of the present invention, but are not intended to limit the scope of the invention. While they are typical of those that might be used, other procedures, methodologies, or techniques known to those skilled in the art may alternatively be used.

### EXAMPLE 1

Materials and Methods

1. Expression and Purification

Expression vectors (pMT/BiP, Invitrogen, Carlsbad, Calif.) were generated by inserting the coding regions for EBOV GP (amino acids 33-647), VP40 (amino acids 1-326) or VP24 (amino acids 1-251) (all sequences are based on Zaire ebolavirus, Mayinga strain, Genbank accession number NC_002549). Drosophila S2 cells adapted to ExCell420 medium (Sigma-Aldrich, St. Louis, Mo.) were co-transformed with expression plasmids and selectable marker plasmid pCoHygro using the calcium phosphate coprecipitation method. Stable transformants were selected by adding hygromycin B to the medium. After selection was complete, cultures of the cell lines were induced by addition of 200 μM CuSO₄ to the culture medium. Expression was verified by SDS-PAGE and western blot. For this, nitrocellulose membranes after western transfer were probed with Ebola hyperimmune mouse ascitic fluid (HMAF) obtained from the US Army Medical Research Institute of Infectious Diseases (USAMRIID), Frederick, Md. This was followed by treatment with a goat anti-mouse IgG alkaline phosphatase-conjugated secondary antibody (Southern Biotech, Birmingham, Ala.) and development with nitro-blue tetrazolium chloride and 5-bromo-4-chloro-3′-indolyl-phosphate (NBT/BCIP; Promega, Madison, Wis.) solid phase alkaline phosphatase substrate. The glycosylation status of the recombinant subunits was documented using either Peptide-N-Glycosidase F (PNGase F; NEB, Ipswich, Me.) to study N-linked glycosylation or a complete enzymatic deglycosylation kit (EDEGLY, Sigma, St. Louis, Mo.) following the manufacturer's instructions.

Antigens were produced in 400 mL spinner flasks or in a WAVE Bioreactor (GE Healthcare, Piscataway, N.J.) using 2 or 10 L bag sizes (and 1-5 L culture volumes) and were subsequently purified by immunoaffinity chromatography (IAC). Monoclonal antibodies specific for the individual proteins (Z-AC1-BG11 (EBOV VP24), M-HD06-A10A (EBOV VP40) and EGP13C6 (EBOV GP)) were obtained from USAMRIID, purified via protein A affinity chromatography and coupled to NHS-Sepharose (GE Healthcare, Piscataway, N.J.) at 10 mg/ml bed volume. For antigen purification, S2 cell culture medium containing recombinant protein was clarified and sterile filtered (0.2 μm pore size). The material was then loaded onto the respective IAC column, at a linear flow rate of approximately 2 cm/min. After the medium was loaded, the matrix was washed with 10 mM phosphate buffered saline, pH 7.2, containing 0.05% (v/v) Tween® 20 (PBST, 140 mM NaCl) followed by washing with 10 mM phosphate buffer, pH 7.2 (no detergent present). Bound protein was eluted from the IAC column with 20 mM glycine buffer, pH 2.5. The eluent was neutralized with 10 mM phosphate buffer, pH 7.2, buffer exchanged into 10 mM phosphate buffered saline, pH7.2 (PBS), and concentrated using Centricon Plus-20 devices (Millipore, Billerica, Mass.). The purified products were analyzed by SDS-PAGE with Coomassie blue or silver staining, western blot, and quantified by UV absorption. Purified recombinant proteins were stored frozen at −80° C. until used for vaccine formulation. The control “NULL” antigen was prepared by concentrating and buffer exchanging supernatants from untransformed S2 cells grown under identical conditions to the S2 cell lines expressing recombinant proteins into PBS using Centricon Plus-20 devices.

2. Mouse Immunogenicity Studies—Vaccine Formulation and Immunization of Mice

All work with animals was conducted in compliance with the Animal Welfare Act and other Federal statutes and regulations relating to animals and experiments involving animals and adhered to the principles stated in the Guide for the Care and Use of Laboratory Animals, NRC Publication, 1996 edition. All procedures were reviewed and approved by the appropriate Institutional Animal Care and Use Committees at the University of Hawaii and USAMRIID. All work with live virus was conducted in the BSL4 animal facility at USAMRIID

For the immunogenicity studies, mice were immunized using four different adjuvants with different modes of action. A saponin-based, TLR-4 (toll-like receptor 4) agonist, GPI-0100 (Hawaii Biotech, Inc., Honolulu, Hi.) [27, 28] was used at doses of 100 or 250 μg. In addition to directly activating the TLR4-pathway, saponins have the ability to modulate immune responses by intercalating into the cell membranes, thus allowing soluble protein antigens to enter the endogenous antigen presentation pathway for “cross presentation” resulting in activation of cytotoxic CD8+ T cells. Three emulsion-based adjuvants were tested: 1) ISA51 (Seppic, Fairfield, N.J.) used at 50% v/v; 2) CoVaccine HT™ (an emulsion of squalane with immunostimulatory sucrose fatty acid sulphate esters and an adjuvant of Protherics Medicines Development Ltd., a BTG Company, London, United Kingdom) [29] used at a dose of 1 mg; and 3) Ribi R-700 (Sigma-Aldrich, St. Louis, Mo.) which in each mouse dose contains 50 μg monophosphoryl lipid A and 50 μg synthetic trehalose dicorynomycolate in a squalene-Tween 80 emulsion. Emulsion-based adjuvants act by sequestering antigens thereby promoting a “depot effect” whereby antigens are slowly released from the depot and provide a longer lasting immune stimulus. In addition, adjuvants containing TLR or PRR (pattern recognition receptor) agonists such as glycans or lipid A may also activate the innate immune system resulting in cytokine release and activation of effector lymphoid cells. Groups of 10 or 15 female BALB/c mice (8 weeks old) were vaccinated subcutaneously (s.c.) three times with individual subunit proteins at the chosen dose level (between 1-10 μg as indicated in the Results section below) and formulated with one of the four selected adjuvants at 4-week intervals. Vaccine formulations were prepared fresh for each vaccination day from frozen antigen stocks, adjuvant stock solutions and sterile PBS to give the desired dose within a final volume of 0.2 mL. Serum samples were obtained 2 weeks after the second vaccination. Five mice from each group were euthanized on the fourth and/or seventh day after the third vaccination and splenectomies were performed for preparation of splenocyte cultures. The remaining five or ten mice from each group were euthanized 14 days after the third vaccination and individual serum samples collected from each animal.

3. Mouse Efficacy Studies

Groups of ten 6 week-old female BALB/c mice were immunized s.c. 3 times at days 0, 28 and 56 with 10 μg doses of VP24, VP40 and/or GP formulated with either 100 μg of GPI-0100 or 1 mg of CoVaccine HT™, or without adjuvant. Negative control groups received equivalent doses of adjuvant only. Serum samples were collected via tail bleeds 2 weeks after each immunization to determine ELISA IgG antibody titers against irradiated EBOV. Approximately one month after the last vaccination, mice were transferred into the BSL4 animal facility and challenged intraperitoneally (i.p.) with 100 pfu of mouse adapted EBOV (ma-EBOV) [30]. Mice were observed daily for signs of illness and death. Surviving animals were euthanized 28 days after challenge.

4. Analysis of Antibodies by ELISA

Sera of individual mice were titrated for IgG specific to the recombinant VP24, VP40 and GP proteins by standard ELISA technique using plates coated with purified recombinant antigens or plates coated with irradiated whole virus [31]. The titers presented are defined as the dilution of antiserum yielding 50% maximum absorbance values (EC₅₀) and was determined using a sigmoidal dose response curve fitting algorithm (Prism, Graphpad Software, San Diego, Calif.). Alternatively, endpoint titers were determined. They were defined as the highest dilution yielding an absorption (A₄₀₅) of 0.2 above background.

5. Proliferation and Cytokine Analysis of Immune Splenocytes

Splenectomies were performed on immunized mice four and/or seven days post final vaccination and splenocyte suspensions prepared. Erythrocytes were lysed with an NH₄Cl solution (0.15 M NH₄Cl, 10 mM KHCO₃, 0.1 mM EDTA, pH 7.3) and the splenocytes were then collected by centrifugation. The resultant cell pellet was washed and resuspended in cell culture medium. Cell counts were performed on each suspension using a cell counter (Beckman Coulter, Brea, Calif.), and the suspensions diluted to 4×10⁶ cells/mL. For proliferation assays, 4×10⁵ splenocytes (0.1 mL) were dispensed into wells of a 96-well cell culture plate. EBOV VP24, VP40 or GP antigens (1 μg/well) in a volume of 0.1 mL were then added to the cell suspensions (in quadruplicate). Unstimulated (antigen omitted) cell suspensions, phytohemagglutinin (PHA, 10 μg/mL, final concentration) stimulated cell suspensions, and “NULL” stimulated cell suspensions (buffer exchanged proteins from S2 cell cultures to document the potential effect of contaminants in antigen preparations) were included as controls. Cultures were incubated at 37° C., 5% CO₂, in humidified chambers for 7 days (3 days for PHA stimulated cultures), and then one microcurie of tritiated (methyl-³H) thymidine (60 Ci/mmol; ICN Biomedicals, Inc., Irvine, Calif.) was added to each well (in a volume of 0.01 mL), and incubation continued for 18 hrs. Cell cultures were harvested onto glass fiber filtration plates (Filtermate Plate Harvester, PerkinElmer Instrument Co., Waltham, Mass.) and analyzed for radioactivity using the TopCount Microplate Scintillation and Luminescence Counter (PerkinElmer Instrument Co., Waltham, Mass.). The stimulation index (SI) was calculated by dividing the specific stimulation counts by the unstimulated cell counts for each suspension. An SI of 3 or greater was considered significant (positive).

For cytokine production assays, 2×10⁶ splenocytes (in 0.5 mL) were dispensed into wells of a 24-well cell culture plate and stimulated with equal volumes of antigens or controls yielding final concentrations of 10⁶ cells/mL and 5 μg/mL of antigen or pokeweed mitogen (instead of PHA) control. Unstimulated controls and “null” antigen controls were also included. The culture supernatants were harvested on day 5 post-stimulation and frozen until analyzed for secreted cytokines. The cytokines interferon-gamma (IFN-γ), tumor necrosis factor-alpha (TNF-α), and interleukins 4, 5, and 10 were assayed by standard ELISA technique or by using a flow cytometric cytokine bead array assay (BD Biosciences, San Jose, Calif.).

6. Passive Protection Studies in BALB/c Mice

Formulations containing 10 μg EBOV GP or VP24 and 1 mg CoVaccine HT™ were administered s.c. three times to groups of 35 female BALB/c mice at 4-week intervals. Fourteen days after the last vaccination, 30 mice from each group were euthanized and serum samples collected by cardiac puncture. Serum samples obtained from each group were pooled and subsequently transferred i.p. to ten naïve BALB/c mice (1.0 mL per mouse). Splenocytes were isolated from the spleens of immunized mice and administered i.p. to groups of ten BALB/c mice (female, 20-25 g) at 7×10⁷ cells/mouse. T-cells were separated from other cell types contained in splenocyte populations by negative selection (using MACs separation technique; Invitrogen, Carlsbad, Calif.). Separated T-cells were administered (i.p.) to naïve mice at rates of 1.5×10⁷ cells/mouse (high dose) and 1.5×10⁶ cells/mouse (low dose). Mice were subsequently transferred into the BSL4 laboratory and challenged approximately 24 hours post serum or cell transfer by i.p. injection with 1000 pfu (30,000 LD₅₀) of ma-EBOV. Survivors were euthanized 28 days post challenge and serum samples collected from selected groups.

7. Statistical Analysis

Significant differences in antibody titers, stimulation indices, or cytokine production between immunized groups of mice were determined by unpaired t tests (GraphPad Prism). P<0.05 was considered to be significant. Significant differences in survival between immunized (or non-immunized control) groups subsequently challenged were determined by the Fisher exact probability test (GraphPad Prism). P<0.05 was considered to be significant.

Results

Expression of Filovirus Immunogens in Drosophila S2 Cells

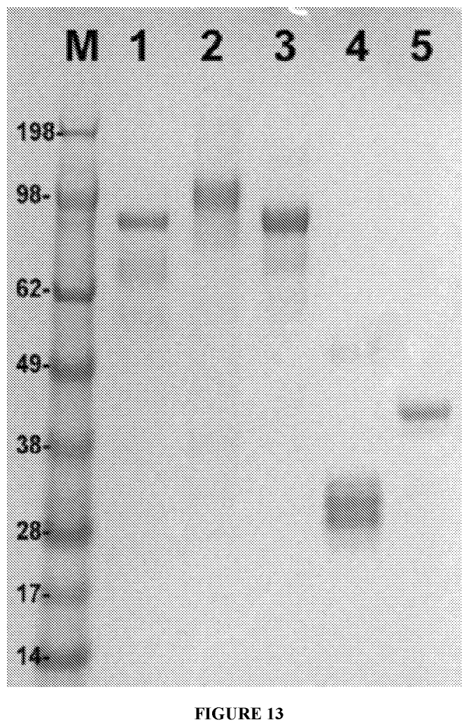

Stably transformed insect cell lines expressed proteins and showed yields between 10-15 mg/L cultured in either spinner flasks or Wave bioreactor. FIG. 12 illustrates the successful expression of secreted Ebola virus subunit proteins. Expression levels were estimated to be >10 μg/ml for all three proteins based on SDS-PAGE gels. GP, VP24 and VP40 antigens were subsequently purified by IAC to 85-95% homogeneity (FIG. 13).

FIG. 12 shows the expression of recombinant EBOV subunits from Drosophila S2 cells. Coomassie stained SDS-PAGE gel (12%) featuring supernatants from Ebola subunit expression lines. Lanes 1, 6 and 11—Molecular weight standard (sizes in kDa), Lanes 2-4: Ebola VP40 (one protein band marked: +), Lanes 7-9: Ebola VP24 (3 bands marked: −), Lanes 13-15: Ebola GP (one protein band marked: *)

FIG. 13 is a gel showing purified recombinant EBOV proteins. 4-12% NuPAGE gel (Invitrogen, Carlsbad, Calif.) loaded with 1 μg each of insect cell expressed, immunoaffinity purified recombinant filovirus proteins. Lane M: Prestained molecular weight marker (Seeblue Plus2, Invitrogen); Lane 1: EBOV GP, Lane 2: MARV GP, Lane 3 SUDV GP, Lane 4: EBOV VP24, lane 5: EBOV VP40. SUDV and MARV GP proteins are expressed using an analogous process to EBOV proteins and are shown here for reference.

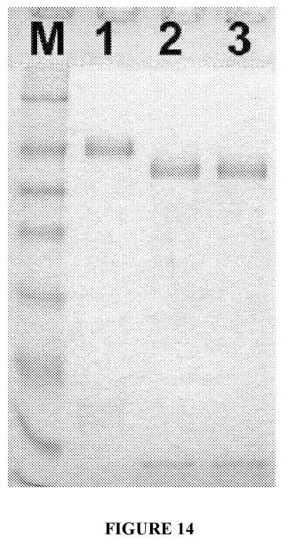

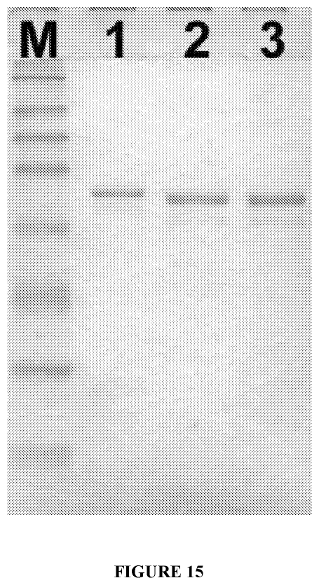

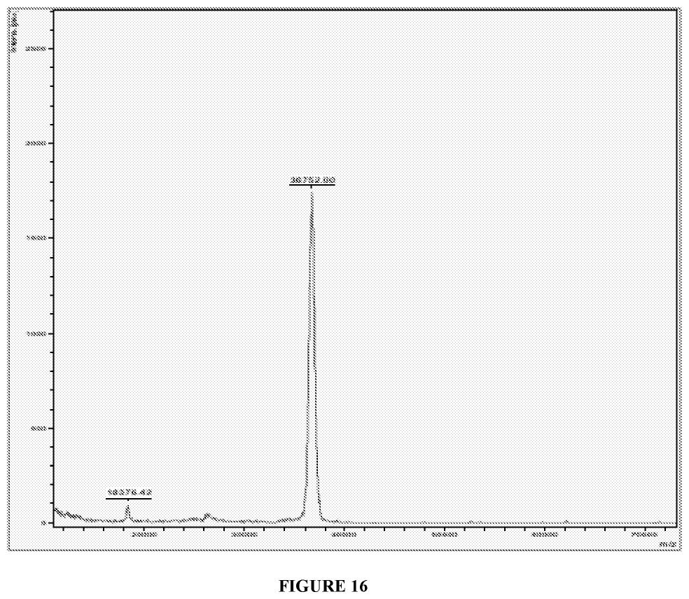

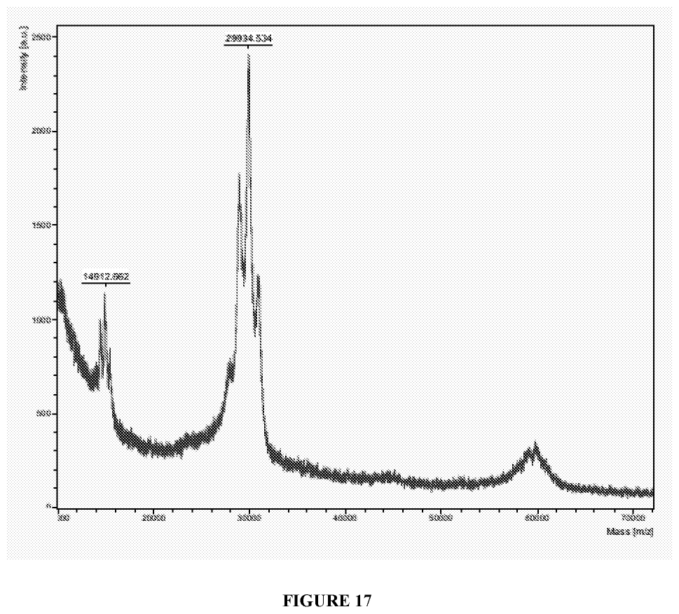

Analysis of the glycosylation status of each of the individual antigens was conducted using enzymatic deglycosylation with analysis on protein gels. For GP, the PNGase treatment resulted in a protein which migrated faster on SDS-PAGE, consistent with the removal of the carbohydrate side chains from all N-linked glycosylation sites (supplementary FIG. 14). In contrast, no evidence was found for O-linked glycosylation using the EDEGLY kit. Reduction of the GP protein results in separation of GP₁ and GP₂ fragments (FIG. 14) and confirms that the furin cleavage site is being processed completely during post-translational processing. PNGase treatment suggests that the VP40 with secretion signal is produced as a uniform product that is glycosylated at one glycosylation site (documented on protein gel, FIG. 15) and by mass spectrometry (FIG. 16). In contrast, VP40 expressed intracellularly is not glycosylated. As expected based on previous work [32], recombinant VP40 in solution shows dimerization as well as higher oligomerization. VP24 contains three internal N-linked glycosylation sites which are partially processed during passage through the secretion pathway resulting in the triplet seen in FIGS. 12 and 13. This finding has also been confirmed by mass spectrometry (FIG. 17).

Recombinant EBOV Antigens Raise Humoral and Cellular Immune Responses in Mice

The purified candidate EBOV immunogens were first used to test their potential in generating humoral and cellular immune responses in BALB/c mice. For this, the three EBOV antigens were tested individually at 10 μg doses in formulations with two functionally different adjuvants, ISA-51 (water-in-oil emulsion) and GPI-0100 (saponin-based preparation). Antibody titers after three vaccinations observed by ELISA using homologous recombinant antigens as coating antigens are shown in FIG. 18.

FIG. 18 is a graphing showing the humoral responses to recombinant EBOV antigens. ELISA IgG antibody titers (EC50) calculated using a sigmoidal dose-response, variable slope program (Graphpad Prism). The GMT+95% CI is plotted for each group (n=5). Plates were coated with the homologous immunizing antigen. Control groups (mice immunized with adjuvant only) were completely negative (EC50 values<<lowest dilution tested, 1:250). Antibody titration curves for all groups including control groups are shown in Figure S5. Differences in antibody titers between GP/GPI-0100 and GP/ISA51 immunized groups were significant (p<0.05). Differences in antibody titers between VP24/GPI-0100 and VP24/ISA51 immunized groups, and between VP40/GPI-0100 and VP40/ISA51 immunized groups were not significant (p>0.05).

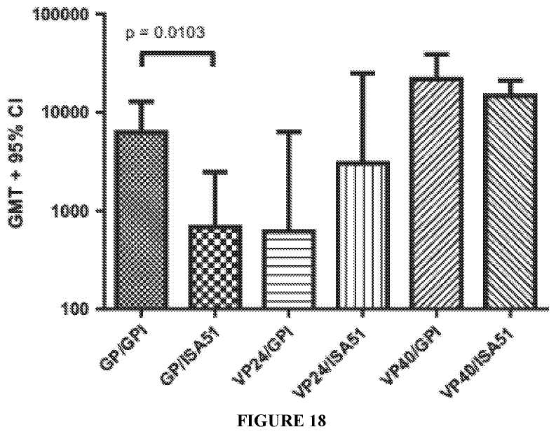

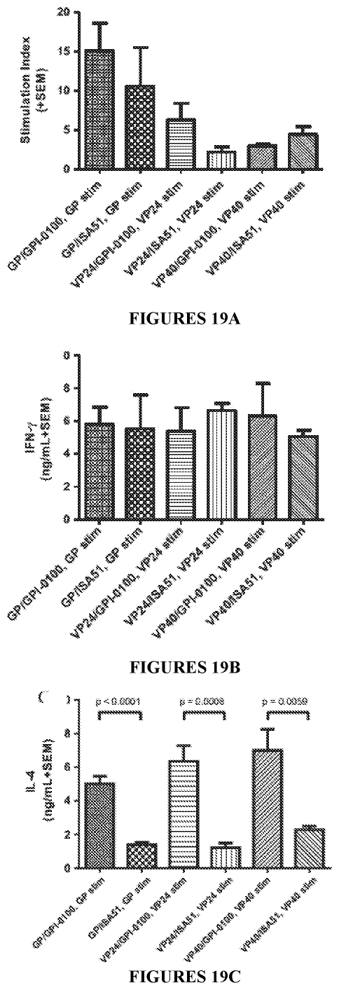

Antibody titers generated against Ebola GP and VP24 were comparatively low after the first vaccination, but increased following the second and third vaccinations (Table 3). In contrast, VP40-specific antibody titers were elicited after only one vaccination and rose above the maximum dilution tested after the third vaccination. Assays for cell-mediated immunity (after three vaccinations) demonstrated that lymphocyte proliferation and IL-4 responses from immune mouse splenocytes were higher in groups administered vaccine formulated with GPI-0100 than with ISA-51 (except for VP40 stimulated proliferation; FIG. 19) as were IL-5 and IL-10 responses. IFN-γ responses were strong in all groups and suggest the ability of the tested antigens to induce potent cell mediated immunity.

FIG. 19 shows graphs depicting cell-mediated immune responses raised by recombinant antigens. Panel A: Mean (n=5 per group) lymphocyte proliferation (indicated as stimulation index, SI) in vitro from immune splenocytes stimulated with homologous antigens. Mean SI from mitogen (PHA) stimulated cultures varied in the range of 4.2-42. Mean SI in splenocyte cultures from adjuvant only immunized mice re-stimulated with GP, VP24, or VP40 was <2.0 in all cases. Panel B: Mean (n=5 per group) IFN-γ production in vitro from immune splenocytes re-stimulated with homologous antigens. Mean IFN-γ production from PWM stimulated cultures varied in the range of 15-58 ng/mL. Mean IFN-γ production from control (unstimulated) cultures was <0.5 ng/mL in all cases. Mean IFN-γ production in splenocyte cultures from adjuvant only immunized mice re-stimulated with GP, VP24, or VP40 was <1.0 ng/mL in all cases. Panel C: Mean (n=5 per group) IL-4 production in vitro from immune splenocytes re-stimulated with homologous antigens. Mean IL-4 production from PWM stimulated cultures varied in the range of 5.7-11.7 ng/mL. Mean IL-4 production from control (unstimulated) cultures was <0.25 ng/mL in all cases. Mean IL-4 production in splenocyte cultures from adjuvant only immunized mice stimulated with GP, VP24, or VP40 was <0.2 ng/mL in all cases. Differences in IL-4 production between groups immunized with formulations containing GPI-0100 or ISA51 were significant (p<0.05) between the groups immunized and re-stimulated using the same antigen. Differences in IFN-γ production or proliferation were not significant (p>0.05) for formulations using the two different adjuvants suggesting that adjuvant has a lower effect on Th1 type responses.

Antigen Dose Response with Selected Adjuvants

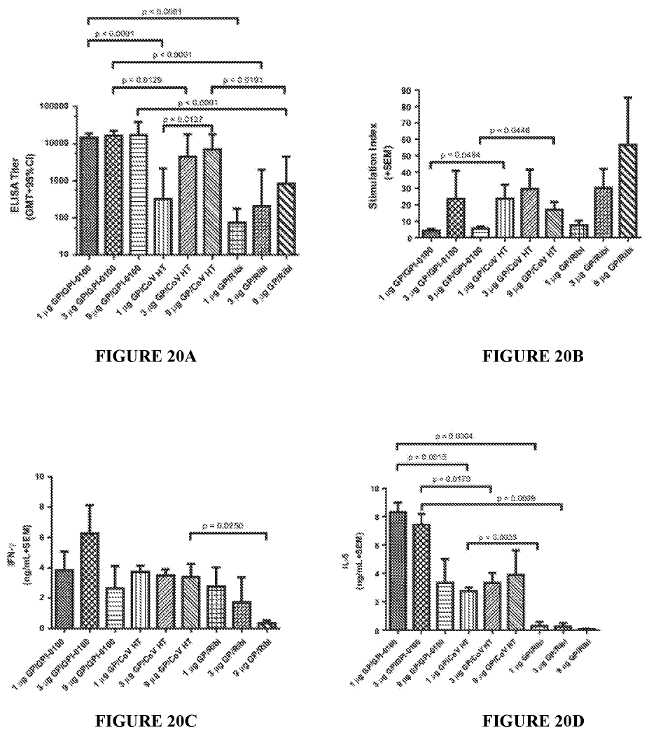

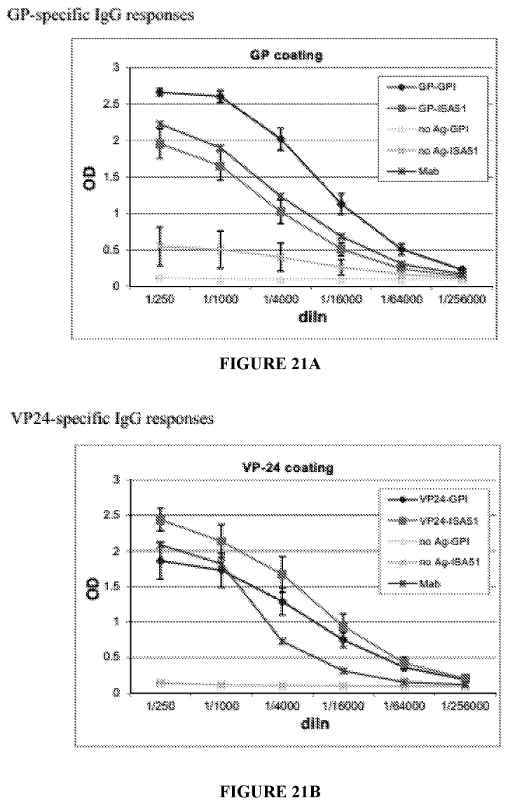

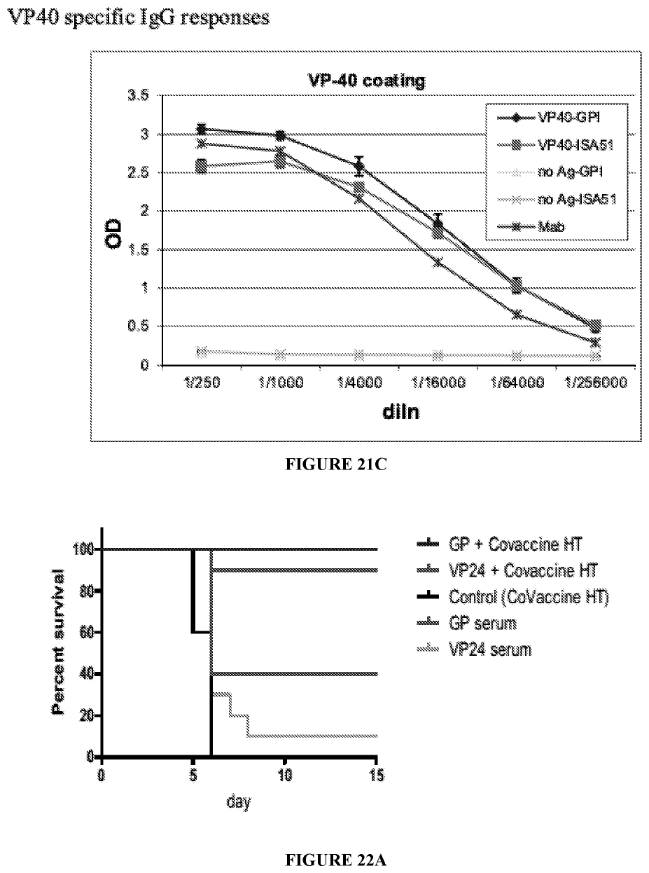

BALB/c mice were immunized with varying amounts of GP antigen to determine the effect of increasing antigen doses on the immune response. The glycoprotein was formulated at three different doses (1, 3, and 9 μg) with GPI-0100, CoVaccine HT™, or Ribi R-700. Results are shown in Table 4 and FIG. 20.

FIG. 20 shows graphs depicting the humoral responses are affected by adjuvant selection and antigen dose. Panel A: ELISA IgG antibody titers post third vaccine dose using plates coated with homologous antigen. EC₅₀ titers from individual animals (n=4 per group) were calculated using a sigmoidal dose-response, variable slope program (Graphpad Prism). The GMT+95% CI is plotted for each group. Significant differences between groups are indicated by overlying horizontal bars on FIG. 5A. At the same antigen dose levels of both 1 or 3 μg of EBOV GP, differences between groups immunized with formulations containing GPI-0100 showed significantly higher titers than formulations containing CoVaccine HT™ (CoV HT) or Ribi. Differences between groups immunized with 9 μg GP and GPI-0100 or Ribi and with 9 μg GP and CoVaccine HT™ or Ribi were also significant (p=0.0191 for both comparisons). IgG titers in formulations containing CoVaccine HT™ showed the only statistically significant dose response when comparing the 1 and 9 μg doses of vaccine. Differences between all other groups were not significant (p>0.05). Panel B: Mean (n=6 per group) lymphocyte proliferation (stimulation index, SI) from immune splenocytes stimulated with homologous antigen in vitro, harvested at day 4 (n=3) or day 7 (n=3) post booster vaccination. Mean SI from mitogen (PHA) stimulated cultures varied in the range of 2.4-50. Mean SI in splenocyte cultures from adjuvant only immunized mice stimulated with GP was <1.7 in all cases. Significant differences between groups are indicated by overlying horizontal bars and showed significant differences between CoVaccine HT™ and GPI-0100 adjuvanted formulations at the 1 and 9 μg GP dose levels. No other pairwise comparisons yielded significant differences. Panel C: Mean (n=3 per group) IFN-γ production in vitro from immune splenocytes stimulated with homologous antigen. Mean IFN-γ production from control (unstimulated) cultures was <0.35 ng/mL in all cases except the 1 μg GP/Ribi group, which had 0.97 ng/mL. Mean IFN-γ production in splenocyte cultures from adjuvant only immunized mice stimulated with GP was undetectable (<0.1 ng/mL) in all cases. Significant differences between groups are indicated by overlying horizontal bars and showed a significant difference only between GPI-0100 and Ribi adjuvanted formulations at the 9 μg GP dose level. No other pairwise comparisons yielded significant differences. Panel D: Mean (n=3 per group) IL-5 production in vitro from immune splenocytes stimulated with homologous antigen. Mean IL-5 production from control (unstimulated) cultures was undetectable (<0.1 ng/mL) in all cases. Mean IL-5 production in splenocyte cultures from adjuvant only immunized mice stimulated with GP was undetectable (<0.1 ng/mL) in all cases. Significant differences between various groups are indicated by overlying horizontal bars. No other pairwise comparisons yielded significant differences.

Similar to the first experiment, antibody responses to GP are relatively low following the first vaccination and all groups immunized with GP showed a typical (increasing) dose-related response following the second vaccination (Table 4). By the third vaccination the titers induced in the GPI-0100 adjuvanted formulation appeared to reach a plateau as dose response was no longer evident, while there was still evidence of a dose response in the groups receiving formulations containing CoVaccine HT™ or Ribi. The GPI-0100 formulation yielded the highest antibody titers, while the Ribi R-700 adjuvanted formulation yielded the lowest antibody titers (FIG. 20A). In general, antigen-stimulated lymphocyte proliferation and cytokine production did not demonstrate consistent antigen dose responses (FIG. 20B-D). With GPI-100 or CoVaccine HT™, there was no antigen dose effect evident at all, with the exception of IL-5 with CoVaccine HT™. With Ribi R-700, there appeared to be a large increase in lymphocyte proliferation between the 1, 3, and 9 μg doses, but these differences were not statistically significant due to the large SEM. In some cases, a decreasing tendency was observed in responses with increasing antigen dose.

Recombinant EBOV Antigens Elicit Protection Against Homologous Challenge with ma-EBOV

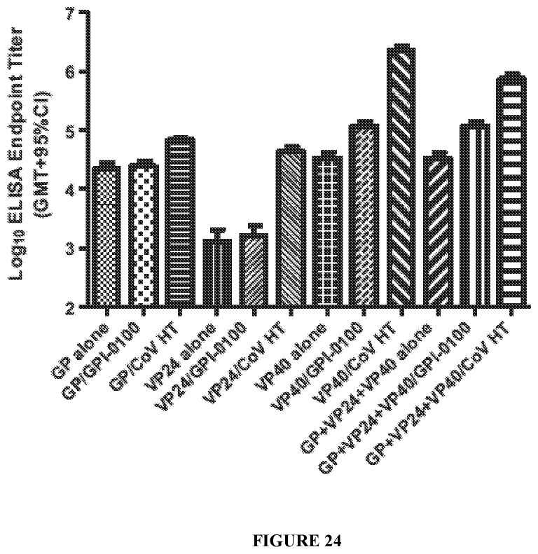

Based on the results of our immunogenicity studies, lead candidate vaccines were formulated using individual recombinant EBOV proteins, or a mixture of all three, for a mouse challenge study. FIG. 24 summarizes these vaccine candidates' immunogenicity based on humoral responses and Table 1 provides the documentation of their protective efficacy. IgG titers verify good immunogenicity of all the proteins, especially in adjuvanted groups.

FIG. 24 shows graphs depicting ELISA IgG antibody titers (endpoint) to irradiated whole virus after three immunizations and prior to virus challenge. Mice were immunized with 10 μg of GP, VP24, VP40, or 10 μg each of GP+VP24+VP40 with and without adjuvants. Log₁₀ antibody titers against irradiated EBOV as coating antigen are shown for all formulations containing antigens. Endpoint titers in control groups (mice immunized with either adjuvant alone) were 1.76 and 2.14 for GPI-0100 and CoVaccine HT™, respectively.

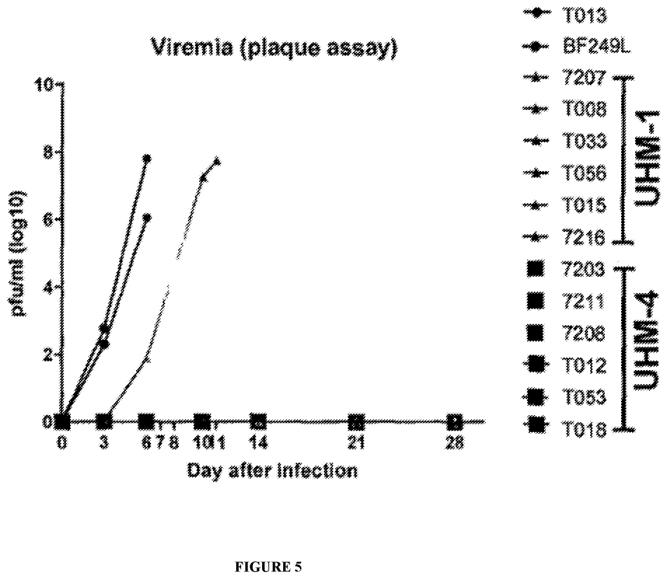

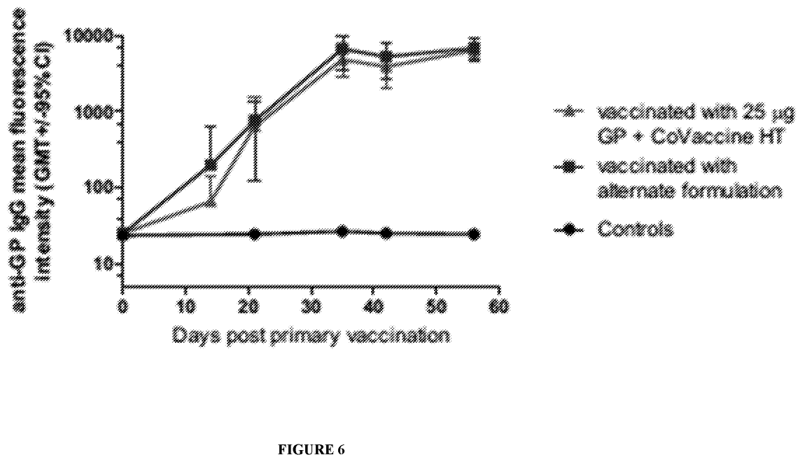

While VP24 antibody titers appear lower, this is due to using irradiated (whole) virus as coating antigen instead of recombinant subunits, as the VP24 antigen is only a minor component of the virus localized inside the particle and thus would not result in as much antibody binding to coating antigen as when animals were immunized with GP or VP40. Formulations containing CoVaccine HT™ induced the highest titers with all antigens and the titers, as previously observed, reached near maximal level after two vaccinations (Table 3). Titers induced by the GPI-0100 based formulations were lower than titers generated by CoVaccine HT™ formulations, but higher than those induced with the unadjuvanted antigens (FIG. 6). Three vaccinations were required to induce maximal titers in mice with either the unadjuvanted or GPI-0100 adjuvanted formulations (Table 3).

Mice were challenged on day 23 after the 3^{rd} vaccination by i.p. injection with ma-EBOV. Morbidity and mortality within individual groups are shown in Table 1. GP alone or formulated with GPI-0100 afforded a high level of protection against mortality but not morbidity. In contrast, GP formulated with CoVaccine HT™ showed 100% protective efficacy against both morbidity and mortality demonstrating the protective potential of the critical GP antigen. The two formulations containing the combination of three antigens co-administered with GPI-0100 or CoVaccine HT™ adjuvant showed full protection against both morbidity and mortality. Surprisingly, immunization of animals with the unadjuvanted antigen combination yielded 90% protective efficacy against morbidity and mortality. Results with unadjuvanted individual proteins generally showed either no protection or a moderate protection level, suggesting a synergistic effect of the combination.

TABLE 1
Recombinant Ebola virus subunits protect mice against live virus
challenge

| | | | | P value vs. |
|---|---|---|---|---|
| | | | Survival | adjuvant |
| Group | | | (day 20 post | control |
| no. | Immunogen^{a} | Adjuvant | challenge) | group^{b} |
| 1 | GP | NONE | 7/10^{c} | 0.0015 |
| 2 | GP | GPI-0100 | 9/10^{c} | 0.0005 |
| 3 | GP | CoVaccine HT ™ | 10/00 | <0.0001 |
| 4 | VP24 | NONE | 0/10 | >0.05 |
| 5 | VP24 | GPI-0100 | 0/10 | >0.05 |
| 6 | VP24 | CoVaccine HT ™ | 6/10^{c} | 0.0054 |
| 7 | VP40 | NONE | 0/10 | >0.05 |
| 8 | VP40 | GPI-0100 | 0/10 | >0.05 |
| 9 | VP40 | CoVaccine HT ™ | 0/10 | >0.05 |
| 10 | GP + VP40 + VP24 | NONE | 9/10 | <0.0001 |
| 11 | GP + VP40 + VP24 | GPI-0100 | 10/10 | <0.0001 |
| 12 | GP + VP40 + VP24 | CoVaccine HT ™ | 10/10 | <0.0001 |
| 13 | NONE | NONE | 0/9 | — |
| 14 | NONE | GPI-0100 | 1/10^{c} | — |
| 15 | NONE | CoVaccine HT ™ | 0/10% | — |

^{a}Mice were immunized with 10 μg of each antigen by the i.m. route;
^{b}Adjuvant control groups: 13 (no adjuvant), 14 (GPI-0100), 15 (CoVaccine HT ™);
^{c}Animals showed signs of illness for part of the study(e.g. ruffled fur).

TABLE 2
Passive transfer of immune serum or immune cells protects naïve
BALB/c mice against lethal challenge.

| Group | n | treatment | Survivors | P value vs. control group |
|---|---|---|---|---|
| 1 | 5 | GP + CoVaccine HT ™ (direct) | 5/5 | 0.0040 |
| 2 | 5 | VP24 + CoVaccine HT ™ (direct) | 2/5 | >0.05 |
| 3 | 5 | CoVaccine HT ™ (direct) | 0/5 | Adjuvant control group |
| 4 | 10 | GP serum (1 ml)^{a} | 9/10 | <0.0001 |
| 5 | 10 | VP24 serum (1 ml)^{a} | 1/10 | >0.05 |
| 6 | 10 | Naïve | 0/10 | Challenge control group |
| 7 | 10 | GP T cells hi (1.5 × 10\hat{ }7)^{b} | 7/10 | p < 0.05^{f} |
| 8 | 10 | VP24 T cells hi (1.5 × 10\hat{ }7)^{b} | 8/10 | p < 0.05^{f} |
| 9 | 10 | GP T cells low (1.5 × 10\hat{ }6)^{c} | 5/10 | p < 0.05^{f} |
| 10 | 10 | VP24 T cells low (1.5 × 10\hat{ }6)^{c} | 5/10 | p < 0.05^{f} |
| 11 | 10 | GP + VP24 T cells (1.5 × 10\hat{ }7 both)^{d} | 8/10 | p < 0.05^{f} |
| 12 | 10 | GP + VP24 T cells (1.5 × 10\hat{ }6 both)^{d} | 6/10 | p < 0.05^{f} |
| 13 | 10 | GP spleno hi (7 × 10\hat{ }7)^{e} | 8/10 | p < 0.05^{f} |
| 14 | 10 | VP24 spleno hi (7 × 10\hat{ }7)^{e} | 5/10 | p < 0.05^{f} |
| 15 | 10 | GP + VP24 spleno hi (7 × 10\hat{ }7 both)^{e} | 8/10 | p < 0.05^{f} |

^{a}1 ml of immune serum per mouse administered i.p.,
^{b}1.5 × 10⁷ T-cells/mouse administered i.p.,
^{c}1.5 × 10⁶ T-cells/mouse administered i.p,.
^{d}mixed cells from group 1 (GP immunized) + group 2 (VP24 immunized) animals; indicated amount of cells administered from both groups into each animal,
^{e}Splenocyte (unfractionated) transfers: 7 × 10⁷ cells/mouse,
^{f}Normal serum, T cell or splenocyte transfers were conducted in the past and have shown that the same amount of normal serum or number of normal T cells or splenocytes administered to mice yield 100% fatalities with the identical challenge virus and dose as administered in this experiment. Thus, all groups of mice receiving anti-GP serum or immune cells in this experiment had significant protection (p < 0.05) compared to mice receiving normal serum or cells.

TABLE 3
Recombinant Ebola virus antigens elicit serum antibody
reactive with homologous antigens and whole virus^{a}

| | | Recombinant antigen^{b} | | | Irradiated Ebola virus^{c} | | |
|---|---|---|---|---|---|---|---|
| | | Post | Post | Post | Post | Post | Post |
| Antigen^{d} | Adjuvant | dose 1 | dose 2 | dose 3 | dose 1 | dose 2 | dose 3 |
| GP | GPI-0100^{e} | 250 | 27,858 | 64,000 | <100 | 1397 | 3363 |
| GP | ISA-51 | <250 | 6964 | 16,000 | <100 | 155 | 580 |
| VP24 | GPI-0100 | <250 | 16,000 | 36,758 | <100 | <100 | <100 |
| VP24 | ISA-51 | <250 | 9190 | 36,758 | <100 | 124 | <100 |
| VP40 | GPI-0100 | 2297 | 147,033 | ≥256,000 | <100 | 37,710 | 30,271 |
| VP40 | ISA-51 | 758 | 48,503 | ≥256,000 | <100 | 15,659 | 30,271 |
| NONE | GPI-0100 | <250 | <250 | <250 | <100 | <100 | <100 |
| NONE | ISA-51 | <250 | <250 | <250 | <100 | <100 | <100 |

^{a}ELISA antibody titers expressed as geometric mean of individual animal serum dilutions yielding an OD of 0.2 above background
^{b}ELISA plates coated with homologous recombinant antigens
^{c}ELISA plates coated with irradiated Ebola virus
^{d}10 μg of each antigen used for immunization by s.c. route
^{e}100 μg of GPI-0100 used

TABLE 4
Summary of ELISA titers from the dose response and adjuvant selection
study ELISA titers are expressed as the dilution yielding the half
maximal absorption value (determined by using a sigmoidal curve fitting
algorithm). Homologous antigen preparations (GP or VP40) from the
same lots as used for immunizations were used as coating antigens.

| | Recombinant GP as | | | Recombinant VP40 as | | |
|---|---|---|---|---|---|---|
| | coating antigen | | | coating antigen | | |
| Vaccine | Post | Post | Post | Post | Post | Post |
| formulation | dose 1 | dose 2 | dose 3 | dose 1 | dose 2 | dose 3 |
| 1 μg GP | <250 | 4097 | 24644 | ND | ND | ND |
| (250 μg GPI-0100) | | | | | | |
| 3 μg GP | <250 | 6215 | 16577 | ND | ND | ND |
| (250 μg GPI-0100) | | | | | | |
| 9 μg GP | <250 | 10403 | 18627 | ND | ND | ND |
| (250 μg GPI-0100) | | | | | | |
| 1 μg GP | <250 | <250 | 1179 | ND | ND | ND |
| (CoVaccine HT ™) | | | | | | |
| 3 μg GP | <250 | 581 | 7588 | ND | ND | ND |
| (CoVaccine HT ™) | | | | | | |
| 9 μg GP | <250 | 1345 | 7297 | ND | ND | ND |
| (CoVaccine HT ™) | | | | | | |
| 1 μg GP | <250 | <250 | <250 | ND | ND | ND |
| (Ribi R-700) | | | | | | |
| 3 μg GP | <250 | <250 | 696 | ND | ND | ND |
| (Ribi R-700) | | | | | | |
| 9 μg GP | <250 | <250 | 2913 | ND | ND | ND |
| (Ribi R-700) | | | | | | |
| 10 μg VP40 | ND | ND | ND | 906 | 22732 | 57638 |
| (250 μg GPI-0100) | | | | | | |
| 10 μg VP40 | ND | ND | ND | 697 | 9023 | 22689 |
| (CoVaccine HT ™) | | | | | | |
| 10 μg VP40 | ND | ND | ND | 157 | 9429 | 24045 |
| (Ribi R-700) | | | | | | |
| Control | <250 | <250 | <250 | <250 | <250 | <250 |
| (250 μg GPI-0100) | | | | | | |
| Control | <250 | <250 | <250 | <250 | <250 | <250 |
| (CoVaccine HT) | | | | | | |
| Control | <250 | <250 | <250 | <250 | <250 | <250 |
| (Ribi R-700) | | | | | | |

(ND: titer not determined).

Protective Efficacy in Mice is Based on Cellular & Humoral Immune Responses

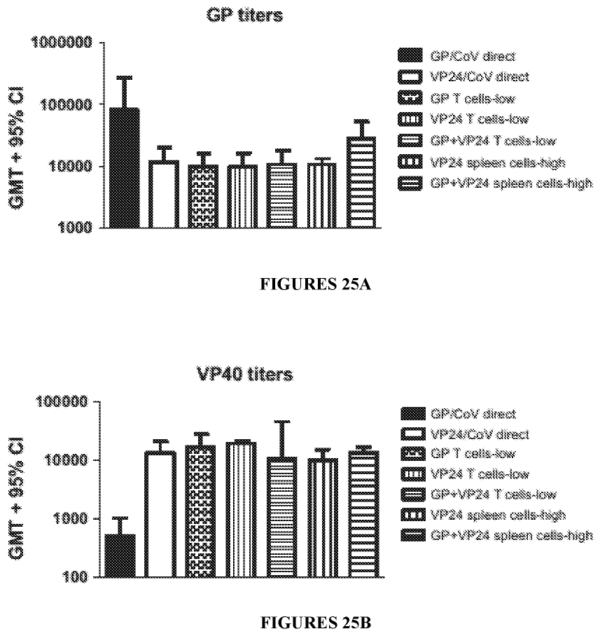

Since individual GP or VP24 subunits were shown to elicit protection in immunized mice, we were interested in identifying the immune mechanisms of protection for these two antigens by performing passive transfer experiments using serum or spleen cells from immunized mice. Pooled anti-GP or anti-VP24 immune sera, whole splenocyte preparations, or isolated T-cells were administered i.p. to naïve BALB/c mice which were challenged approximately 24 hours later. Pre-challenge sera analyzed for antigen specific ELISA IgG titers showed GMT (EC50 titers) >100,000 for both antigens after two or three vaccinations. Direct challenge controls verified previous findings of full protection in GP-vaccinees and partial protection in animals receiving the VP24-only formulation (Table 2). Survivors were euthanized 28 days post challenge and serum samples collected from selected groups. Post-challenge antibody titers to GP and VP40 in survivors are shown in FIG. 25.

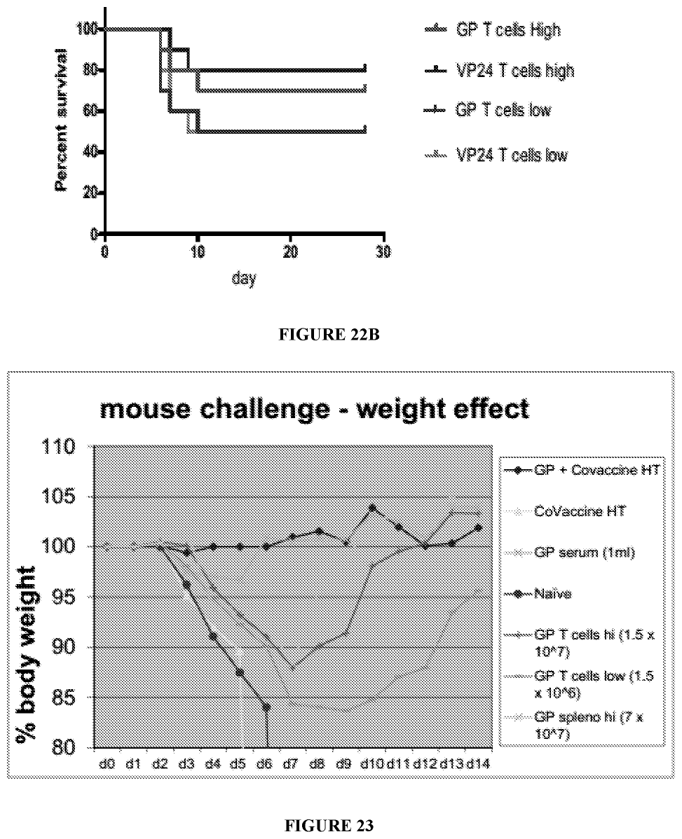

As expected, transfer of GP-specific antiserum produced near complete protection in naive recipients, while VP24-specific serum did not (Table 2; selected Kaplan-Meier survival plots are shown in FIG. 22).

Protected animals receiving GP-specific serum and the directly challenged GP-vaccinees showed no weight loss (FIG. 23), an indicator of morbidity in the model. Post-challenge ELISA analysis was performed as induction of GP and VP40-specific IgG responses in the naive recipients may indicate viral replication.

FIG. 25 shows IgG ELISA antibody titers (EC₅₀) against recombinant EBOV GP and VP40 after live virus challenge in the passive protection experiment. Serum samples of all surviving animals in selected groups were collected at the end of the study and after irradiation analyzed for IgG titers against EBOV GP and VP40 (individually). Panel A: Antibody titers to GP antigen. Panel B: Antibody titers to VP40 antigen.

Anti-GP ELISA titers in serum from directly challenged mice remained steady (FIG. 25A), while post challenge anti-VP40 titers observed (FIG. 25B) were extremely low suggesting that no or only minimal viral replication occurred. Isolated T-cells as well as whole splenocyte preparations protected the majority of naive recipients from death. For T-cell transfer a dose-dependency was seen for individual and mixed cell populations. The post-challenge serum samples showed equivalent IgG titers against both antigens in all groups of immune cell adoptees but one: animals receiving whole mixed splenocytes developed considerably higher anti-GP titers. This result is very likely due to activation of GP-specific memory B-cells that are part of the whole splenocyte preparation. In summary, this experiment demonstrated that recombinant GP as well as VP24 not only induce potent humoral responses, but also generate functional cellular immune responses in T-cells as well that confer protection against viral challenge.

Discussion

Expression of the recombinant EBOV antigens from Drosophila S2 cells yielded high quality protein secreted into the culture medium. GP appears as a single band product indicating complete processing of its (N-linked) glycosylation sites and the furin cleavage site is processed completely leading to separation of GP1 and GP2 regions upon reduction of disulfide linkages. Despite an absence of O-linked glycosylations, the purified recombinant GP demonstrates excellent immunogenic properties and also reacts with EBOV GP specific antibodies in convalescent serum or serum from immunized rodents and primates. In contrast to the proteins present in virus infected cells, the intrinsic glycosylation sites of recombinant VP24 and VP40 are processed either partially (at three sites for VP24) or uniformly (at one site for VP40) during secretion into the culture supernatant. Nevertheless, these post-translational modifications of the proteins did not affect purification using IAC methods, their reactivity with antigen-specific antibodies from convalescent serum samples, or immunogenic potential. This eliminates the need for cell lysis and allows for use of IAC as a gentler purification method that protects native conformation of the antigens.

The use of recombinant proteins as vaccine antigens is a standard approach for contemporary vaccine development. However, in the filovirus field some earlier setbacks in experiments with inactivated viruses [24] or recombinant proteins [33] had a significant impact on application of recombinant subunits to the formulation of vaccine candidates. Expression yields of full length GP in mammalian cells are typically poor (in the range of 1 mg/L when transiently expressed from transfected cells) and purification may be problematic due to the amount of contaminants relative to target protein and the diversity of protein species achieved via processing of O-linked glycosylation sites. More recent approaches therefore use mammalian cell expressed GP fused to the Fc fragment of human IgG1 [34] or, similarly, a plant expressed Ebola Immune Complex (EIC) composed of human or murine antibodies and the GP1 region of EBOV GP [35]. Both of these chimeric antigens can be purified using standard affinity chromatography methods for immunoglobulins. GP expression from Sf9 cells infected with recombinant baculoviruses has been used as an alternative to generate fully glycosylated GP. While the MARV and EBOV GP's derived from baculovirus expression, in conjunction with Ribi® R-700 adjuvant, have shown good immunogenicity in guinea pigs, only a moderate level of protection in the guinea pig models of Marburg and Ebola Hemorrhagic Fever was reported [33, 36, 37]. In contrast, our studies show that the IAC-purified Drosophila-expressed GP does not only result in significant humoral responses in BALB/c mice, but three vaccinations with antigen induced 70% protection, even in the absence of an adjuvant. This level of protection in mice is close to the 80% efficacy reported for another recombinant subunit approach using EIC [38]. While the EIC approach utilized a similar dose level (10 μg), four immunizations and the use of an adjuvant were required to achieve this level of efficacy. With proper adjuvantation (e.g., using CoVaccine HT™) three 10 μg doses of the Drosophila expressed GP completely protected mice from ma-EBOV challenge, a result replicated in two experiments shown herein. Full protection in the mouse model has been met by all leading EBOV vaccine candidates and the immunogenicity data generated suggests that the GP antigen produces robust humoral responses over a wide dose range and that the responses can be enhanced by adjuvants with diverse modes of action. Cell-mediated responses against GP are more variable and careful adjuvant selection will be required to optimize these.

The immunogenicity of purified VP24 and VP40 subunits was strong, and while the adjuvant chosen had a significant impact on final antibody titers observed, the cell mediated responses were robust in all tested formulations. The immunogenicity of the recombinant VP40 is extraordinary, most likely linked to its propensity to assemble donut-shaped hexamers, nanoparticles which could be observed upon electron microscopic evaluation of concentrated supernatants from Drosophila cells expressing VP40 (data not shown). Therefore, given the abundance of VP40 in viral particles, it was a surprise that none of the 30 VP40 vaccinees infected with ma-EBOV survived the challenge (Table I), especially since Wilson et al. [20] reported partial protection when alphavirus replicons expressing VP40 were administered and Olinger et al identified CTL responses to VP40 [21]. This may be linked to a difference in antigen presentation and it would therefore be important to compare which cell types are primarily targeted by the two different approaches as well as by VLP's which have been reported to directly activate dendritic cells [39, 40].

Mice immunized with VP24 in CoVaccine HT™ showed a relatively consistent percentage of survival after challenge (6/10 and 2/5, Tables I and II), although surviving animals showed clear signs of disease pathology (e.g., ruffled fur, abnormal gait, lethargy). As expected based on its localization and excellent ability to raise cell-mediated responses as indicated by cytokine release after antigen restimulation, the protective effect of VP24 is mediated by T-cell immunity as demonstrated by passive (adoptive) transfer studies here and previously using replicons [21]. This mechanism of action should be further investigated as it potentially provides insight into potential therapies to alleviate the effects of EVD.

A combination of all three recombinant antigens in the absence of adjuvant was able to protect 9/10 mice not only from mortality but also from overt EBOV-associated morbidity. The kinetics of antibody response and the ultimate titers achieved (against irradiated EBOV) were not significantly different from those found in animals immunized with GP only. These observations suggest that VP24 and VP40 induce cell-mediated responses that develop a synergy in enhancing the quality of the protective response. As expected, clinical adjuvants raised the efficacy level to 100% and therefore our vaccine candidate of GP with CoVaccine HT™ as well as the combination of three antigens with adjuvants yield equivalent or superior responses to those seen with EBOV VLPs in mice [41]. While a role of VP24 in protection has already been identified based on adoptive transfer of immunity with T cells, additional mechanistic studies will be required to determine if T-cells primed with recombinant VP40 also contribute to protection. Furthermore, assessing the compartmentalization of T cell responses (i.e., CD4+ or CD8+ T-cells) may help to elucidate if VP24 mainly induces T helper cells or also cytotoxic T cell responses aiding in viral clearance. The ability to fine-tune the immune responses against the individual vaccine components is one of the advantages of applying a deliberate mix of non-replicating virus subunits and can facilitate more mechanistic studies as required for dissection of the mechanism of protection afforded by this or similar vaccine candidates.

Filoviruses induce a disease in the immune system of primates in which the symptomatic (hemorrhagic) phase is primarily a secondary reaction to a dysregulated immune response [42]. The current knowledge of EBOV pathogenesis has been reviewed in detail by Falasca et al. [43]. However, the mechanisms of how filoviruses evade the immune system or, most importantly, why the few survivors develop an immune response protecting them from death are still poorly understood. In human cases a correlation was made which indicated that patients with an IgM response maintained for a long period of time had a lower chance of survival than patients who showed a faster maturation towards IgG responses [4]. A potential explanation could be a lack or delay of IL-12 responses from virus-infected monocyte-derived dendritic cells [44] which would have an impact on development of helper T cells and subsequently delay the maturation of the antibody response. EBOV infection of monocytes and macrophages has in contrast been shown to actually increase activation of pro-inflammatory cytokine responses [45] and may therefore delay development of adaptive responses. The answer to the question of why innate mechanisms of protection cannot clear the virus may lie within the components of EBOV that seem to mislead or suppress the immune system, for example due to the presence of soluble glycoprotein (sGP) and truncation variants of the mature GP [46]. EBOV infection also induces apoptosis in primary antigen-presenting cells which unquestionably slows down the host's ability to mount an adaptive response. By contact with macrophages and monocytes, filoviruses appear to trigger inflammatory responses independent of virus replication [45] that ultimately can cause hemorrhage and death of the primate host. One possible explanation for this may be the presence of an immunosuppressive region (mucin-like domain) identified in the GP [47]. In addition to possible effects linked to GP, VP35 [48, 49] and VP24 [50, 51] have both been shown to act as potent inhibitors of IFN type 1 signaling. Mice infected by wild type EBOV show normal IFN-signaling, enabling a protective immune response to develop [52]. In contrast, ma-EBOV inhibits type I interferon stimulated antiviral responses causing increased virulence in mice. This increased virulence may possibly be related to mutations observed in VP24 and NP of ma-EBOV [53]. Similarly, the lower virulence of RESTV compared to EBOV (or MARV) could also be linked to the level of inhibition of type I interferon responses [54], based on a genomic analysis of the host responses in EBOV infected primates.

While the efficacy data of the rVSV-ZEBOV vaccine candidate are impressive, safety of this vaccine is one of the main concerns reported by Huttner et al. [3], who examined the effects of vaccine dose on safety and immunogenicity in a phase 1/2 clinical trial. Three dose levels of vaccine were evaluated: 3×10⁵, 1×10⁷, and 5×10⁷ pfu and safety was assessed by reactogenicity using multiple parameters. After administering the two higher doses of vaccine to 51 subjects, viral oligoarthritis was observed in 11 of them. At that point the studies with the two higher doses were stopped and only the lowest dose level continued. While there was less reactogenicity observed at the lowest dose, the immunogenicity was also decreased in that there was a significant drop in antibody titers at the lowest dose compared to the higher doses. It should be pointed out that the dose demonstrating efficacy by Henao-Restrepo et al. [1, 2] in the Guinea ring vaccination trial was 2×10⁷ pfu. While the identification of a protective antibody titer has not been determined, it is likely that higher antibody titers would yield better efficacy. This is of high relevance in this context, as a recombinant subunit vaccine could further be used to design a successful prime-boost approach, enhancing the fast onset of immunity of a virally vectored vaccine candidate with a consistent boost of IgG titers and increased durability of protection.

In summary, the data presented in this EXAMPLE suggests that a carefully designed vaccine candidate based on recombinant virus subunits can be used to effectively elicit protective responses which allows the host to battle the arsenal of “molecular weapons” which the Ebola virus deploys to stifle the immune system while maintaining a desirable safety profile.

### EXAMPLE 2

Protective Efficacy in Rhesus Macaques may be Adjuvant-Dependent

Guided by the results obtained in mice and guinea pigs and from preliminary non-human primate work, alum was selected as the preferred adjuvant for an efficacy study in rhesus macaques. Recombinant GP and VP24 were adsorbed to aluminum hydroxide (Alhydrogel, Brenntag) and administered three times at 3-week intervals using 50 μg doses. Another experimental group was treated with the optimized antigen mix in CoVaccine HT administered at a 25 μg antigen dose level guided by earlier testing in primates. This study was conducted in collaboration with IRF Frederick and Rocky Mountain Laboratories (both NIAID/NIH). Challenge results are shown in Table 5. All vaccinees developed virus neutralizing antibody titers in the range of 20-40 (group 1) or 40-80 (group 2).

Two of the animals receiving the candidate formulation with EBOV antigens in CoVaccine HT™ were protected against challenge with 1000 LD50's of EBOV (Kikwit strain). While one of these animals showed signs of extremely low level viral replication (viremia<2 logs of genome equivalents/mL only detectable for one day by PCR, virus culture was negative), the second survivor remained completely aviremic by both test methods. However, both animals showed anamnestic responses indicated by a rapid rise in GP- and VP24-specific IgG titers to post dose 2 levels and by virus neutralizing titers maintained at a moderate level (1:80) or increased (from 1:40 up to 1:640) after challenge. The two animals from the same group that were euthanized showed lower pre-challenge anti-GP IgG titers (but similar virus neutralizing titers). However, a rapid depletion of anti-GP IgG can be seen after challenge in these animals, but not in survivors. Interestingly, both of the vaccinated survivors showed significantly higher GP-specific IgM titers than fatalities after each immunization and also after challenge.

The promising results achieved with alum-adsorbed subunits in guinea pigs were not replicated in the rhesus model, however, CoVaccine HT™ adjuvanted formulations (at the 2.5 mg dose level) showed promise. We speculate that immunogenicity with alum may be enhanced in guinea pigs due to the hypersensitivity of these animals to aluminum salts or possibly an inadequate amount of alum being injected into macaques requiring further analysis. However, it is interesting that animals that succumbed to infection all showed a rapid depletion in GP-specific IgG titers suggesting the importance of re-activation of B-cell memory in survival and the importance of a proper adjuvant in generating such an adaptive immune response.

### EXAMPLE 3

Protective Efficacy in Cynomolgus Macaques is Adjuvant-Dependent

These experiments were conducted using the “FANG” challenge model (F) with 100 pfu of 7U low passage virus (testing of candidates UHM-1, 2, 3) with challenge at TBRI or USAMRIID, or the Geisbert model (G) with 1000 pfu of 7U low passage virus (for testing of candidates UHM-1, UHM-4, UHM-5) where testing occurred at UTMB. Three doses of vaccine were administered in 3-week intervals followed by challenge after 4 weeks.

Table 6—Summary of challenge results in cynomolgus macaques (Grey shaded fields: significant protection). IgG titers against the three EBOV antigens using three different adjuvants.

Summary: While antibody titers to GP and other EBOV antigens are observed in all vaccinated animals, only the formulation containing CoVaccine HT™ consistently reaches the highest titers and is the only adjuvant that induces protective efficacy.

Formulation

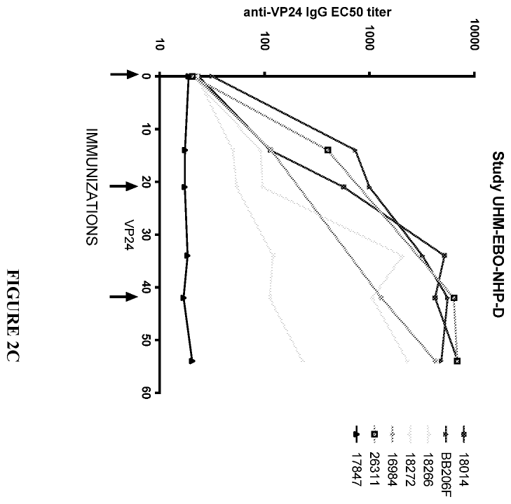

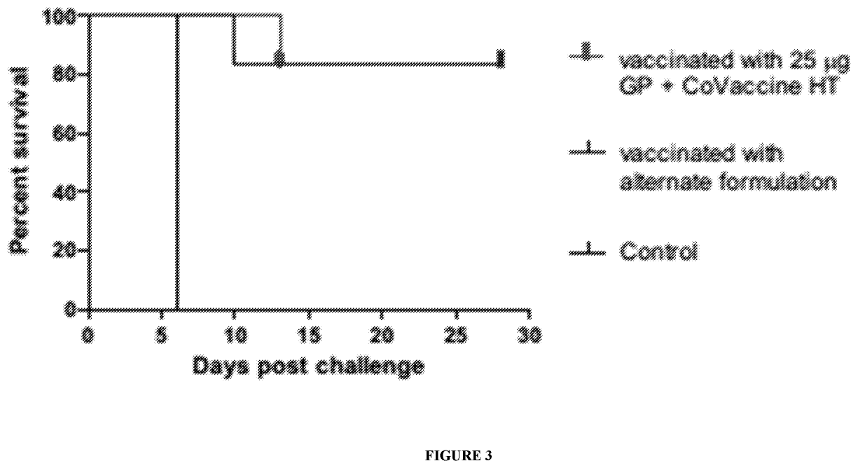

Cynomolgus macaques (Macaca fascicularis) were chosen for conduct of a non-human primate immunogenicity and efficacy experiment using the EBOV challenge model developed by Dr. Thomas Geisbert (Galveston National Laboratory/UTMB). This experiment used animals of both sexes and older (5-15 years old) than typically used for EBOV challenge studies found in the literature (typically 3-4 years old). We believe that this better reflects a representative age distribution than basing development only on young adults. One group of animals was immunized by the intramuscular route (IM) three times at three week intervals with 25 μg of EBOV GP formulated with 10 mg of CoVaccine HT™ adjuvant, a second group was immunized with an alternate formulation (containing GP with recombinant EBOV VP24 and VP40 proteins), while the control group was given only adjuvant. Four weeks after the last vaccination, all animals were challenged by the subcutaneous route (SC) with 1000 LD50 of EBOV, strain Kikwit (7U isolate 199510621, stock number R4414 (Kugelman et al. 2016). Animals were monitored twice daily for morbidity and mortality for up to 28 days. Results are given in Table 7 below and survival curves are shown in FIG. 3. FIG. 3 shows survival of vaccinated and control monkeys after EBOV challenge. Using either the Log-rank (Mantel-Cox) test or the Gehan-Breslow-Wilcoxon test, both of the curves for the vaccinated animals are significantly different from the controls (p=0.0082).

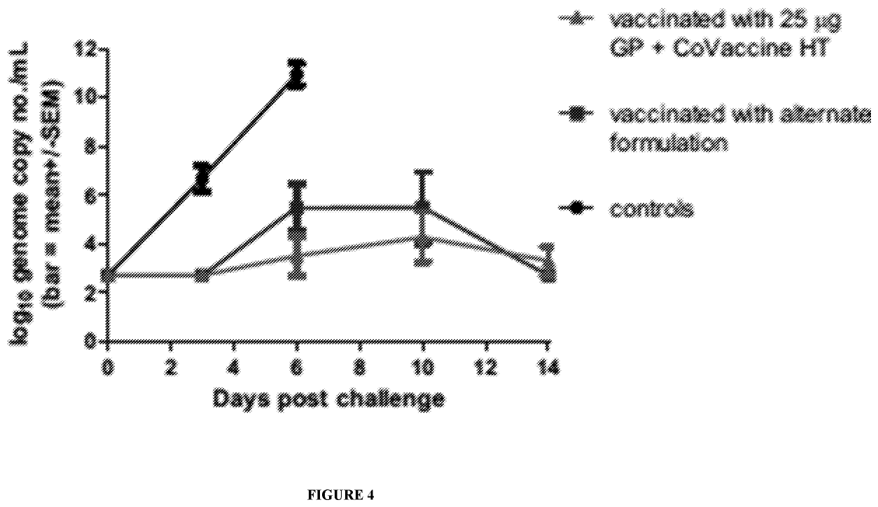

Viremia was determined by rt-PCR and plaque assay. Sera from all animals were collected at 3-4 day intervals until death or day 28 (survivors). The results are shown in FIG. 4. FIG. 4 shows kinetics of viremia for 14 days after challenge. Viremia was determined by rt-PCR on serum samples from individual animals—Limit of detection: 3 log₁₀. The data demonstrate the inhibition of viremia as a result of vaccination with UHM-4. The animals vaccinated with UHM-1 showed slightly higher virus load than animals vaccinated with GP+CoVaccine HT.

FIG. 5 shows viremia post challenge by plaque assay. Viral plaques were only observed in both control animals, and with a significant delay in one animal immunized with the formulation UHM-1. This demonstrates dramatically how vaccination with the recombinant subunit monovalent Ebola vaccine completely protects all vaccinees from infection with Ebola Virus.

Antibody titers were determined on serum samples from vaccinated animals at various time points post vaccination but prior to challenge. The results shown in FIG. 6 demonstrate a robust humoral immune response. There is no statistically significant difference between titers elicited by either vaccine formulation.

FIG. 6 depicts IgG antibody titers to Ebola GP antigen determined by the MIA assay on vaccinated animals. Animals were immunized on days 0, 21, and 42. Antibody levels in vaccinated animals rose rapidly after the first and second immunizations and reached a plateau by 14 days post dose 2 (day 35).

The results of the NHP efficacy study demonstrated full vaccine protection against live EBOV challenge, successful inhibition of viremia, and high antibody titers following vaccination with potent titers after two doses.

As shown above, protection with the recombinant subunit candidate has only been shown using CoVaccine HT adjuvant. Safety and immunogenicity in both primate species tested were excellent.

TABLE 7
Results from EBOV challenge study in cynomolgus macaques

| | | # survivors/total |
|---|---|---|
| | | # of animals |
| Group | Vaccine composition | challenged |
| 1 | UHM-4 (25 μg EBOV GP + | 5/6^{a} |
| | 10 mg CoVaccine HT | |
| | adjuvant) | |
| 2 | UHM-1 (25 μg GP, 25 μg | 5/6 |
| | VP24, 5 μg VP40 + 10 mg | |
| | CoVaccine HT adjuvant) | |
| 3 | Adjuvant only | 0/2 |

^{a}The single animal that met the euthanasia criteria in group 1 was a 15-year-old male and did not show any signs of Ebola Virus Disease (EVD) (based on clinical chemistry and the necropsy report). The animal that had to be euthanized in group 2 was also a 15-year-old male who showed some clinical markers of EVD.

### EXAMPLE 4

Immunogenicity of Thermostabilized EBOV GP Antigen in Mice.

Tests of the thermostabilized EBOV GP protein demonstrate stability at accelerated conditions (40° C.) for at least 4 weeks. Groups of 10 Swiss Webster outbred 7-8 week old mice were immunized by the intramuscular (i.m.) route three times at 3 week intervals with the following formulations:
1) Covaccine HT™ adjuvant alone
2) EBOV GP antigen (liquid) without adjuvant
3) EBOV GP antigen (liquid) with CoVaccine HT™ HT adjuvant
4) EBOV GP antigen (liquid) after incubation at 25° C. for 4 weeks with CoVaccine HT™ adjuvant
5) EBOV GP antigen (liquid) after incubation at 40° C. for 4 weeks with CoVaccine HT™ adjuvant
6) EBOV GP antigen (lyophilized) without adjuvant
7) EBOV GP antigen (lyophilized) with CoVaccine HT™ adjuvant
8) EBOV GP antigen (lyophilized) after incubation at 25° C. for 4 weeks with CoVaccine HT™ adjuvant
9) EBOV GP antigen (lyophilized) after incubation at 40° C. for 4 weeks with CoVaccine HT™ adjuvant

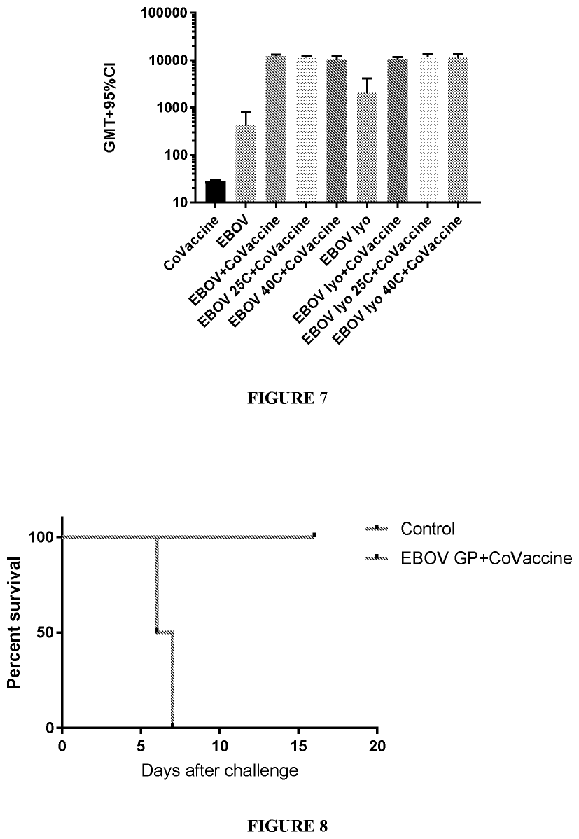

All mice were bled fourteen days after the last vaccination and antibody (IgG) titers were measured in individual mouse sera by a multiplex bead-based immunoassay (Luminex) against the EBOV GP antigen. The results are depicted below in FIG. 7. The geometric mean titer (GMT+95% confidence interval [CI]) of the individual mouse titers (as median fluorescence intensity (MFI) in the Luminex assay) is plotted for each group. The results demonstrate that the immunogenicity of the lyophilized preparation is at least as good as the liquid antigen, and that both preparations are stable for up to four weeks at temperatures as high as 40° C. EBOV GP Vaccination: Efficacy in Non-Human Primates

Animals vaccinated with EBOV GP were completely protected from lethal EBOV infection with 100% (6/6) survival, vs. 0% (0/2) in the controls (p<0.05 contingency, p<0.05 comparison of survival curves) (olate EBOV (7U Kikwit strain).

This data is to our knowledge the first report of a protective Ebola vaccine based on a recombinant subunit protein.

Three doses of vaccine (25 μg EBOV GP (liquid)+10 mg CoVaccine HT) were administered at 3-week intervals intramuscularly to a group of 6 cynomolgus macaques (3 males and 3 females). Control animals (1 male and 1 female) received adjuvant only. Animals were challenged intra-muscularly 28 days after the last vaccination with low-passage, human isolate EBOV (7U Kikwit strain).

FIG. 8: Non-human primate survival after vaccination with EBOV GP and challenge with live EBOV (low passage 7U variant of the Kikwit strain).

### EXAMPLE 5

Monovalent MARV GP Vaccination and Bivalent EBOV GP+MARV GP Vaccination—Efficacy in Non-Human Primates Against MARV Challenge

The data show that animals vaccinated with either MARV GP alone or in combination with EBOV GP protein (BiFiloVax liquid) were completely protected from lethal MARV infection with 100% (4/4) survival, vs. 0% (0/2) in the controls (p<0.05 contingency, p<0.05 comparison of survival curves) (FIG. 3). This data has just been obtained in June 2018 and is to our knowledge the first recombinant subunit vaccine that is 100% effective against MARV challenge. The addition of EBOV GP to the MARV GP in the vaccine did not affect the protection against MARV infection. (p<0.05 contingency combining vaccine groups, p<0.05 comparison of survival curves).

Groups of 4 cynomolgus macaques (2 males and 2 females) were given three doses of either 25 μg MARV GP+10 mg CoVaccine HT™ or 25 μg EBOV GP+25 MARV GP+10 mg CoVaccine HT™ (BiFilovax liquid), at 3-week intervals, while controls (1 male and 1 female) received adjuvant only. Animals were challenged intra-muscularly 28 days after the last vaccination with live MARV.

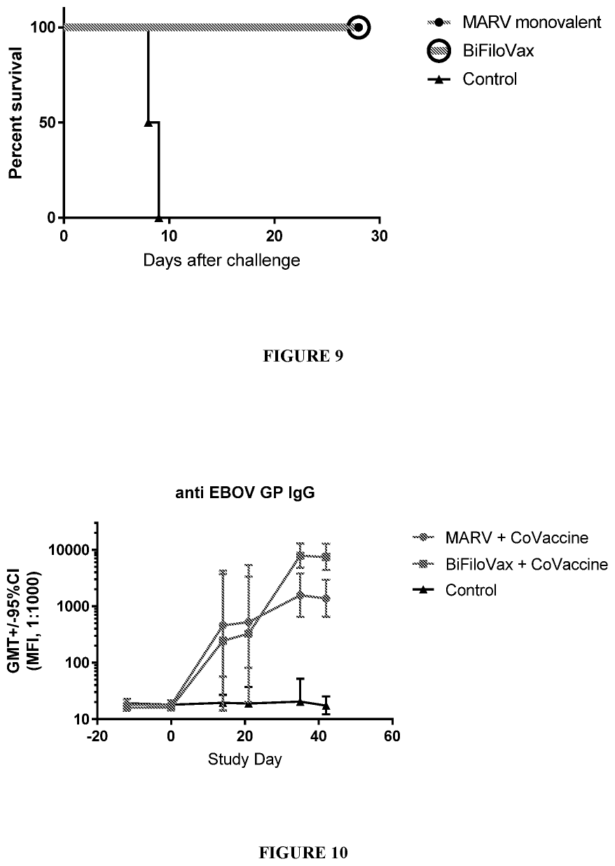

FIG. 9: Non-human primate survival after vaccination with MARV GP or MARV+EBOV GP (BiFiloVax liquid) and challenge with live MARV (low passage Angola strain).

Immunogenicity of Vaccine Formulations in NHP

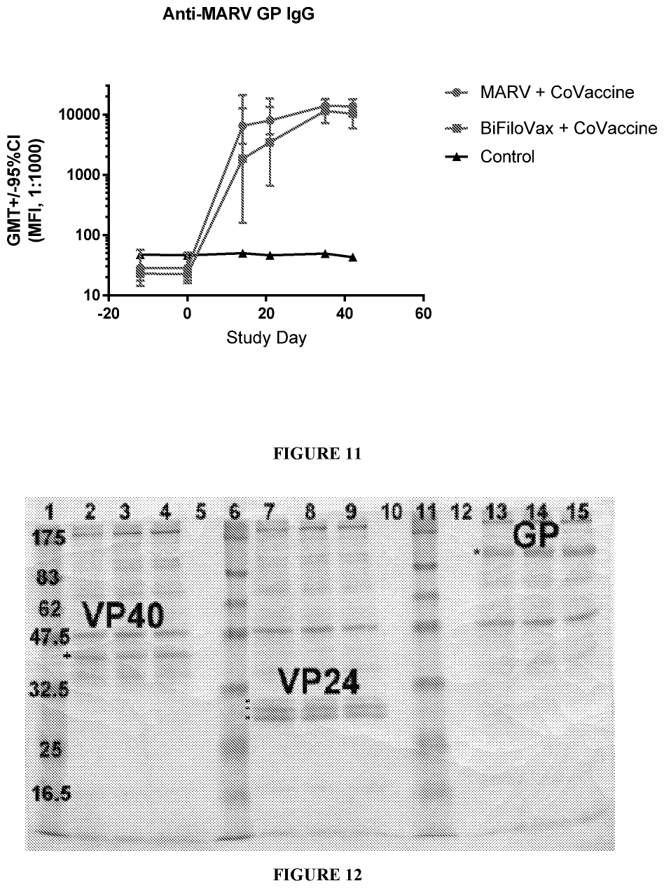

Immunogenicity assessments from the NHP study demonstrate that the bivalent vaccine formulation engenders high titers of antibodies to both antigens at equivalent levels in NHP after two doses of vaccine. Antibody levels to both EBOV and MARV GP were determined by the MIA assay and the results are shown in FIGS. 9-11.

### REFERENCES

[1] Henao-Restrepo, A. M., Camacho, A., Longini, I. M., Watson, C. H., Edmunds, W. J., Egger, M., Carroll, M. W., Dean, N. E., Diatta, I., Doumbia, M., Draguez, B., Duraffour, S., Enwere, G., Grais, R., Gunther, S., Gsell, P. S., Hossmann, S., Watle, S. V., Konde, M. K., Keita, S., Kone, S., Kuisma, E., Levine, M. M., Mandal, S., Mauget, T., Norheim, G., Riveros, X., Soumah, A., Trelle, S., Vicari, A. S., Rottingen, J. A., and Kieny, M. P., Efficacy and effectiveness of an rVSV-vectored vaccine in preventing Ebola virus disease: final results from the Guinea ring vaccination, open-label, cluster-randomised trial (Ebola Ca Suffit!). Lancet, 2016.
[2] Henao-Restrepo, A. M., Longini, I. M., Egger, M., Dean, N. E., Edmunds, W. J., Camacho, A., Carroll, M. W., Doumbia, M., Draguez, B., Duraffour, S., Enwere, G., Grais, R., Gunther, S., Hossmann, S., Konde, M. K., Kone, S., Kuisma, E., Levine, M. M., Mandal, S., Norheim, G., Riveros, X., Soumah, A., Trelle, S., Vicari, A. S., Watson, C. H., Keita, S., Kieny, M. P., and Rottingen, J. A., Efficacy and effectiveness of an rVSV-vectored vaccine expressing Ebola surface glycoprotein: interim results from the Guinea ring vaccination cluster-randomised trial. Lancet, 2015. 386(9996): p. 857-66.
[3] Huttner, A., Dayer, J. A., Yerly, S., Combescure, C., Auderset, F., Desmeules, J., Eickmann, M., Finckh, A., Goncalves, A. R., Hooper, J. W., Kaya, G., Krahling, V., Kwilas, S., Lemaitre, B., Matthey, A., Silvera, P., Becker, S., Fast, P. E., Moorthy, V., Kieny, M. P., Kaiser, L., Siegrist, C. A., and Consortium, V. S.-E., The effect of dose on the safety and immunogenicity of the VSV Ebola candidate vaccine: a randomised double-blind, placebo-controlled phase 1/2 trial. Lancet Infect Dis, 2015. 15(10): p. 1156-66.
[4] Ksiazek, T. G., Rollin, P. E., Williams, A. J., Bressler, D. S., Martin, M. L., Swanepoel, R., Burt, F. J., Leman, P. A., Khan, A. S., Rowe, A. K., Mukunu, R., Sanchez, A., and Peters, C. J., Clinical virology of Ebola hemorrhagic fever (EHF): virus, virus antigen, and IgG and IgM antibody findings among EHF patients in Kikwit, Democratic Republic of the Congo, 1995. J Infect Dis, 1999. 179 Suppl 1: p. S177-87.
[5] Mupapa, K., Massamba, M., Kibadi, K., Kuvula, K., Bwaka, A., Kipasa, M., Colebunders, R., and Muyembe-Tamfum, J. J., Treatment of Ebola hemorrhagic fever with blood transfusions from convalescent patients. International Scientific and Technical Committee. J Infect Dis, 1999. 179 Suppl 1: p. S18-23.
[6] Qiu, X., Audet, J., Wong, G., Fernando, L., Bello, A., Pillet, S., Alimonti, J. B., and Kobinger, G. P., Sustained protection against Ebola virus infection following treatment of infected nonhuman primates with ZMAb. Sci Rep, 2013. 3: p. 3365.
[7] Khurana, S., Fuentes, S., Coyle, E. M., Ravichandran, S., Davey, R. T., Jr., and Beigel, J. H., Human antibody repertoire after VSV-Ebola vaccination identifies novel targets and virus-neutralizing IgM antibodies. Nat Med, 2016. 22(12): p. 1439-1447.
[8] Noda, T., Sagara, H., Suzuki, E., Takada, A., Kida, H., and Kawaoka, Y., Ebola virus VP40 drives the formation of virus-like filamentous particles along with GP. J Virol, 2002. 76(10): p. 4855-65.
[9] Takada, A., Ebihara, H., Jones, S., Feldmann, H., and Kawaoka, Y., Protective efficacy of neutralizing antibodies against Ebola virus infection. Vaccine, 2007. 25(6): p. 993-9.
[10] Jones, S. M., Feldmann, H., Stroher, U., Geisbert, J. B., Fernando, L., Grolla, A., Klenk, H. D., Sullivan, N. J., Volchkov, V. E., Fritz, E. A., Daddario, K. M., Hensley, L. E., Jahrling, P. B., and Geisbert, T. W., Live attenuated recombinant vaccine protects nonhuman primates against Ebola and Marburg viruses. Nat Med, 2005. 11(7): p. 786-90.
[11] Sullivan, N. J., Geisbert, T. W., Geisbert, J. B., Xu, L., Yang, Z. Y., Roederer, M., Koup, R. A., Jahrling, P. B., and Nabel, G. J., Accelerated vaccination for Ebola virus haemorrhagic fever in non-human primates. Nature, 2003. 424(6949): p. 681-4.
[12] Herbert, A. S., Kuehne, A. I., Barth, J. F., Ortiz, R. A., Nichols, D. K., Zak, S. E., Stonier, S. W., Muhammad, M. A., Bakken, R. R., Prugar, L. I., Olinger, G. G., Groebner, J. L., Lee, J. S., Pratt, W. D., Custer, M., Kamrud, K. I., Smith, J. F., Hart, M. K., and Dye, J. M., Venezuelan equine encephalitis virus replicon particle vaccine protects nonhuman primates from intramuscular and aerosol challenge with ebolavirus. J Virol, 2013. 87(9): p. 4952-64.
[13] Bukreyev, A., Rollin, P. E., Tate, M. K., Yang, L., Zaki, S. R., Shieh, W. J., Murphy, B. R., Collins, P. L., and Sanchez, A., Successful topical respiratory tract immunization of primates against Ebola virus. J Virol, 2007. 81(12): p. 6379-88.
[14] Marzi, A., Ebihara, H., Callison, J., Groseth, A., Williams, K. J., Geisbert, T. W., and Feldmann, H., Vesicular stomatitis virus-based Ebola vaccines with improved cross-protective efficacy. J Infect Dis, 2011. 204 Suppl 3: p. S1066-74.
[15] Xu, L., Sanchez, A., Yang, Z., Zaki, S. R., Nabel, E. G., Nichol, S. T., and Nabel, G. J., Immunization for Ebola virus infection. Nat Med, 1998. 4(1): p. 37-42.
[16] Sullivan, N. J., Sanchez, A., Rollin, P. E., Yang, Z. Y., and Nabel, G. J., Development of a preventive vaccine for Ebola virus infection in primates. Nature, 2000. 408(6812): p. 605-9.
[17] Warfield, K. L., Posten, N. A., Swenson, D. L., Olinger, G. G., Esposito, D., Gillette, W. K., Hopkins, R. F., Costantino, J., Panchal, R. G., Hartley, J. L., Aman, M. J., and Bavari, S., Filovirus-like particles produced in insect cells: immunogenicity and protection in rodents. J Infect Dis, 2007. 196 Suppl 2: p. S421-9.
[18] Simmons, G., Lee, A., Rennekamp, A. J., Fan, X., Bates, P., and Shen, H., Identification of murine T-cell epitopes in Ebola virus nucleoprotein. Virology, 2004. 318(1): p. 224-30.
[19] Wilson, J. A. and Hart, M. K., Protection from Ebola virus mediated by cytotoxic T lymphocytes specific for the viral nucleoprotein. J Virol, 2001. 75(6): p. 2660-4.
[20] Wilson, J. A., Bray, M., Bakken, R., and Hart, M. K., Vaccine potential of Ebola virus VP24, VP30, VP35, and VP40 proteins. Virology, 2001. 286(2): p. 384-90.
[21] Olinger, G. G., Bailey, M. A., Dye, J. M., Bakken, R., Kuehne, A., Kondig, J., Wilson, J., Hogan, R. J., and Hart, M. K., Protective cytotoxic T-cell responses induced by venezuelan equine encephalitis virus replicons expressing Ebola virus proteins. J Virol, 2005. 79(22): p. 14189-96.
[22] Warfield, K. L., Swenson, D. L., Olinger, G. G., Kalina, W. V., Aman, M. J., and Bavari, S., Ebola virus-like particle-based vaccine protects nonhuman primates against lethal Ebola virus challenge. J Infect Dis, 2007. 196 Suppl 2: p. S430-7.
[23] Ignatyev, G. M., Agafonov, A. P., Streltsova, M. A., and Kashentseva, E. A., Inactivated Marburg virus elicits a nonprotective immune response in Rhesus monkeys. J Biotechnol, 1996. 44(1-3): p. 111-8.
[24] Geisbert, T. W., Pushko, P., Anderson, K., Smith, J., Davis, K. J., and Jahrling, P. B., Evaluation in nonhuman primates of vaccines against Ebola virus. Emerg Infect Dis, 2002. 8(5): p. 503-7.
[25] Modis, Y., Ogata, S., Clements, D., and Harrison, S. C., A ligand-binding pocket in the dengue virus envelope glycoprotein. Proc Natl Acad Sci USA, 2003. 100(12): p. 6986-91.
[26] Modis, Y., Ogata, S., Clements, D., and Harrison, S. C., Structure of the dengue virus envelope protein after membrane fusion. Nature, 2004. 427(6972): p. 313-9.
[27] Marciani, D. J., Press, J. B., Reynolds, R. C., Pathak, A. K., Pathak, V., Gundy, L. E., Farmer, J. T., Koratich, M. S., and May, R. D., Development of semisynthetic triterpenoid saponin derivatives with immune stimulating activity. Vaccine, 2000. 18(27): p. 3141-51.
[28] Marciani, D. J., Reynolds, R. C., Pathak, A. K., Finley-Woodman, K., and May, R. D., Fractionation, structural studies, and immunological characterization of the semi-synthetic Quillaja saponins derivative GPI-0100. Vaccine, 2003. 21(25-26): p. 3961-71.
[29] Hilgers, L. A. and Blom, A. G., Sucrose fatty acid sulphate esters as novel vaccine adjuvant. Vaccine, 2006. 24 Suppl 2: p. S2-81-2.
[30] Bray, M., Davis, K., Geisbert, T., Schmaljohn, C., and Huggins, J., A mouse model for evaluation of prophylaxis and therapy of Ebola hemorrhagic fever. J Infect Dis, 1998. 178(3): p. 651-61.
[31] Ksiazek, T. G., West, C. P., Rollin, P. E., Jahrling, P. B., and Peters, C. J., ELISA for the detection of antibodies to Ebola viruses. J Infect Dis, 1999. 179 Suppl 1: p. S192-8.
[32] Timmins, J., Schoehn, G., Ricard-Blum, S., Scianimanico, S., Vernet, T., Ruigrok, R. W., and Weissenhorn, W., Ebola virus matrix protein VP40 interaction with human cellular factors Tsg101 and Nedd4. J Mol Biol, 2003. 326(2): p. 493-502.
[33] Mellquist-Riemenschneider, J. L., Garrison, A. R., Geisbert, J. B., Saikh, K. U., Heidebrink, K. D., Jahrling, P. B., Ulrich, R. G., and Schmaljohn, C. S., Comparison of the protective efficacy of DNA and baculovirus-derived protein vaccines for EBOLA virus in guinea pigs. Virus Res, 2003. 92(2): p. 187-93.
[34] Konduru, K., Bradfute, S. B., Jacques, J., Manangeeswaran, M., Nakamura, S., Morshed, S., Wood, S. C., Bavari, S., and Kaplan, G. G., Ebola virus glycoprotein Fc fusion protein confers protection against lethal challenge in vaccinated mice. Vaccine, 2011. 29(16): p. 2968-77.
[35] Phoolcharoen, W., Bhoo, S. H., Lai, H., Ma, J., Arntzen, C. J., Chen, Q., and Mason, H. S., Expression of an immunogenic Ebola immune complex in Nicotiana benthamiana. Plant Biotechnol J, 2011. 9(7): p. 807-16.
[36] Hevey, M., Negley, D., Geisbert, J., Jahrling, P., and Schmaljohn, A., Antigenicity and vaccine potential of Marburg virus glycoprotein expressed by baculovirus recombinants. Virology, 1997. 239(1): p. 206-16.
[37] Hevey, M., Negley, D., VanderZanden, L., Tammariello, R. F., Geisbert, J., Schmaljohn, C., Smith, J. F., Jahrling, P. B., and Schmaljohn, A. L., Marburg virus vaccines: comparing classical and new approaches. Vaccine, 2001. 20(3-4): p. 586-93.
[38] Phoolcharoen, W., Dye, J. M., Kilbourne, J., Piensook, K., Pratt, W. D., Arntzen, C. J., Chen, Q., Mason, H. S., and Herbst-Kralovetz, M. M., A nonreplicating subunit vaccine protects mice against lethal Ebola virus challenge. Proc Natl Acad Sci USA, 2011. 108(51): p. 20695-700.
[39] Bosio, C. M., Moore, B. D., Warfield, K. L., Ruthel, G., Mohamadzadeh, M., Aman, M. J., and Bavari, S., Ebola and Marburg virus-like particles activate human myeloid dendritic cells. Virology, 2004. 326(2): p. 280-7.
[40] Ye, L., Lin, J., Sun, Y., Bennouna, S., Lo, M., Wu, Q., Bu, Z., Pulendran, B., Compans, R. W., and Yang, C., Ebola virus-like particles produced in insect cells exhibit dendritic cell stimulating activity and induce neutralizing antibodies. Virology, 2006. 351(2): p. 260-70.
[41] Warfield, K. L., Bosio, C. M., Welcher, B. C., Deal, E. M., Mohamadzadeh, M., Schmaljohn, A., Aman, M. J., and Bavari, S., Ebola virus-like particles protect from lethal Ebola virus infection. Proc Natl Acad Sci USA, 2003. 100(26): p. 15889-94.
[42] Geisbert, T. W. and Jahrling, P. B., Exotic emerging viral diseases: progress and challenges. Nat Med, 2004. 10(12 Suppl): p. S110-21.
[43] Falasca, L., Agrati, C., Petrosillo, N., Di Caro, A., Capobianchi, M. R., Ippolito, G., and Piacentini, M., Molecular mechanisms of Ebola virus pathogenesis: focus on cell death. Cell Death Differ, 2015. 22(8): p. 1250-9.
[44] Bosio, C. M., Aman, M. J., Grogan, C., Hogan, R., Ruthel, G., Negley, D., Mohamadzadeh, M., Bavari, S., and Schmaljohn, A., Ebola and Marburg viruses replicate in monocyte-derived dendritic cells without inducing the production of cytokines and full maturation. J Infect Dis, 2003. 188(11): p. 1630-8.
[45] Stroher, U., West, E., Bugany, H., Klenk, H. D., Schnittler, H. J., and Feldmann, H., Infection and activation of monocytes by Marburg and Ebola viruses. J Virol, 2001. 75(22): p. 11025-33.
[46] Escudero-Perez, B., Volchkova, V. A., Dolnik, O., Lawrence, P., and Volchkov, V. E., Shed GP of Ebola virus triggers immune activation and increased vascular permeability. PLoS Pathog, 2014. 10(11): p. e1004509.
[47] Yaddanapudi, K., Palacios, G., Towner, J. S., Chen, I., Sariol, C. A., Nichol, S. T., and Lipkin, W. I., Implication of a retrovirus-like glycoprotein peptide in the immunopathogenesis of Ebola and Marburg viruses. FASEB J, 2006. 20(14): p. 2519-30.
[48] Basler, C. F., Mikulasova, A., Martinez-Sobrido, L., Paragas, J., Muhlberger, E., Bray, M., Klenk, H. D., Palese, P., and Garcia-Sastre, A., The Ebola virus VP35 protein inhibits activation of interferon regulatory factor 3. J Virol, 2003. 77(14): p. 7945-56.
[49] Basler, C. F., Wang, X., Muhlberger, E., Volchkov, V., Paragas, J., Klenk, H. D., Garcia-Sastre, A., and Palese, P., The Ebola virus VP35 protein functions as a type I IFN antagonist. Proc Natl Acad Sci USA, 2000. 97(22): p. 12289-94.
[50] Reid, S. P., Valmas, C., Martinez, O., Sanchez, F. M., and Basler, C. F., Ebola virus VP24 proteins inhibit the interaction of NPI-1 subfamily karyopherin alpha proteins with activated STAT1. J Virol, 2007. 81(24): p. 13469-77.
[51] Reid, S. P., Leung, L. W., Hartman, A. L., Martinez, O., Shaw, M. L., Carbonnelle, C., Volchkov, V. E., Nichol, S. T., and Basler, C. F., Ebola virus VP24 binds karyopherin alphal and blocks STAT1 nuclear accumulation. J Virol, 2006. 80(11): p. 5156-67.
[52] Bray, M., The role of the Type I interferon response in the resistance of mice to filovirus infection. J Gen Virol, 2001. 82(Pt 6): p. 1365-73.
[53] Ebihara, H., Takada, A., Kobasa, D., Jones, S., Neumann, G., Theriault, S., Bray, M., Feldmann, H., and Kawaoka, Y., Molecular determinants of Ebola virus virulence in mice. PLoS Pathog, 2006. 2(7): p. e73.
[54] Kash, J. C., Muhlberger, E., Carter, V., Grosch, M., Perwitasari, O., Proll, S. C., Thomas, M. J., Weber, F., Klenk, H. D., and Katze, M. G., Global suppression of the host antiviral response by Ebola- and Marburgviruses: increased antagonism of the type I interferon response is associated with enhanced virulence. J Virol, 2006. 80(6): p. 3009-20.

Although the invention has been described with reference to the above example, it will be understood that modifications and variations are encompassed within the spirit and scope of the invention. Accordingly, the invention is limited only by the following claims.

What is claimed is:

1. A composition comprising at least one filovirus glycoprotein (GP) formulated with an adjuvant, wherein the adjuvant comprises a sucrose fatty acid sulphate ester, wherein the composition elicits an immune response when administered to a subject, which response is protective upon challenge with a filovirus.

2. The composition of claim 1, wherein the filovirus is selected from Zaire Ebolavirus (EBOV), Sudan Ebolavirus (SUDV) or Marburgvirus (MARV).

3. The composition of claim 1, wherein the adjuvant comprises a physiological salt solution, or an oil-in-water emulsion, or a water immiscible solid phase, and optionally an aqueous phase, and comprising, as an adjuvant, one or more disaccharide derivatives of formula: 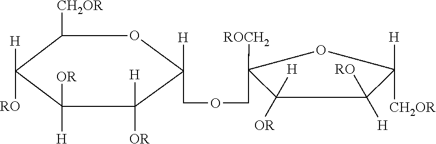
wherein
(i) at least 3, but not more than N−1, of the groups R are represented by: —C(═O)—(CH₂)ₓCH₃ groups, wherein x is between 6 and 14, and
(ii) at least one, but no more than N−1, of the groups R are anionic —SO₂—OR¹ groups, wherein R¹ is a monovalent cation,
wherein N is the number of groups R of the disaccharide derivative and wherein the combined number of —C(═O)—(CH₂)ₓCH₃ and —SO₂—OR¹ groups does not exceed N and the remaining groups R are hydrogen.

4. The composition of claim 3, wherein the disaccharide derivative has no more than N−2, or no more than N−3, anionic —SO₂—OR¹ groups.

5. 5The composition of claim 3, wherein the disaccharide derivative has at least 4, but no more than N−1, —C(═O)—(CH2)XCH3 groups and no more than N−3, or no more than N−4, anionic —SO2-OR1 groups.

6. The composition of claim 3, wherein the disaccharide derivative has two, three or four anionic —SO₂—OR¹ groups, and at least three —C(═O)—(CH₂)ₓCH₃ groups, wherein the total sum of anionic —SO₂—OR¹ groups and —C(═O)—(CH₂)ₓCH₃ groups is in the range of about 6 or 7.

7. The composition of claim 3, wherein the monovalent cation is independently selected from the group consisting of H⁺, K⁺, Na⁺, Li⁺ and NH₄⁺.

8. The composition of claim 3, which comprises an oil in water emulsion, wherein said oil-in-water emulsion comprises a water-immiscible liquid phase which is squalane, a mineral oil, a plant oil, hexadecane, a fluorocarbon or a silicon oil.

9. The composition of claim 8, further comprising an emulsifier or stabilizer.

10. The composition of claim 9, wherein the emulsifier or stabilizer is a non-ionic detergent with a hydrophilic-lipophilic balance value of more than 10, a sugar fatty acid ester, or an anionic detergent with a hydrophilic-lipophilic balance value of more than 10.

11. The composition of claim 9, wherein the emulsifier or stabilizer is a disaccharide derivative.

12. The composition of claim 8, wherein the water immiscible solid phase is an insoluble salt.

13. The composition of claim 8, wherein the insoluble salt is an aluminum or calcium salt, preferably an aluminum hydroxide, aluminum phosphate, calcium phosphate, silica or a mixture thereof.

14. The composition of claim 3, wherein the adjuvant is CoVaccineHT™.

15. The composition of claim 1, further comprising at least one matrix protein.

16. The composition of claim 15, wherein the matrix proteins are filovirus VP24 and/or VP40.

17. A method of inducing a protective immune response to infection with a filovirus comprising administering to a subject in need thereof, a protective effective amount of a composition of claim 1, thereby protecting the subject from infection with the filovirus.

18. The method of claim 17, wherein the filovirus is selected from Zaire Ebolavirus (EBOV), Sudan Ebolavirus (SUDV) or Marburgvirus (MARV).

19. The method of claim 17, wherein the subject is a human.

20. The method of claim 17, wherein upon administration, the subject develops antibody titers.

21. The method of claim 20, wherein the antibodies are IgG or IgM.

22. The method of claim 17, wherein administration is in one or more immunizations.

23. The method of claim 17, wherein the adjuvant of claim 1, comprises a physiological salt solution, or an oil-in-water emulsion, or a water immiscible solid phase, and optionally an aqueous phase, and comprising, as an adjuvant, one or more disaccharide derivatives of formula: 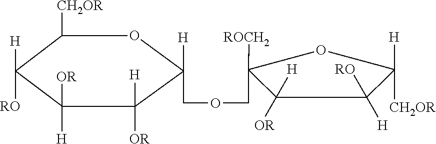
wherein
(i) at least 3, but not more than N−1, of the groups R are represented by: —C(═O)—(CH₂)ₓCH₃ groups, wherein x is between 6 and 14, and
(ii) at least one, but no more than N−1, of the groups R are anionic —SO₂—OR¹ groups, wherein R¹ is a monovalent cation,
wherein N is the number of groups R of the disaccharide derivative and wherein the combined number of —C(═O)—(CH₂)ₓCH₃ and —SO₂—OR¹ groups does not exceed N and the remaining groups R are hydrogen.

24. The method of claim 23, wherein the adjuvant is CoVaccineHT™.

25. The composition of claim 1, wherein the GP is at least one or a combination of GPs from Zaire Ebolavirus (EBOV), Sudan Ebolavirus (SUDV) or Marburgvirus (MARV).

26. The composition of claim 25, further comprising at least one non-Filovirus antigen.
